SEQUENCE LISTING (72 sequences)

SEQ ID NO: 1 - DNA, 876 bp, Vigna angularis
atgggttcttctttgtggacttgtctgattctgttatcactggcttctgcttctttcgctgccaacccaagaactccaattgatgtaccatttggcagaaactatgtgcctacttgggcctttgatcatatcaaatatctcaatggaggttctgagattcagcttcatctcgataagtacactggtactggattccagtccaaagggtcatacttgtttggtcacttcagcatgtacataaaattggttcctggtgattcagctggcacagtcactgctttctatttatcgtccacaaacgcagaacatgatgaaatagacttcgagttcttgggaaacagaactgggcaaccatacattttacaaacaaatgtgttcaccggaggcaaaggtgacagagagcagagaatctacctctggtttgaccctacgactcaataccacagatattcagtgctatggaacatgtaccagattgtattctatgtggatgactacccaataagggtgttcaagaacagcaatgacttgggagtgaagttccccttcaatcaaccaatgaaaatatacaacagtttgtggaatgcagatgactgggctacaaggggtggtttggagaaaacagattggtccaaagcccccttcatagcctcttacaagggcttccacattgatgggtgtgaggcctcagtgaatgccaagttctgtgacacacaaggcaagaggtggtgggatcaaccagagtttcgtgaccttgatgctgctcagtggcaaaaactggcttgggtacgcaacaaatacaccatctacaactactgcactgatcgcaaacgctactctcaagtccctccagagtgcaccagagaccgtgacatt

SEQ ID NO: 2 - protein, 292 aa, Vigna angularis
MGSSLWTCLILLSLASASFAANPRTPIDVPFGRNYVPTWAFDHIKYLNGGSEIQLHLDKYTGTGFQSKGSYLFGHFSMYIKLVPGDSAGTVTAFYLSSTNAEHDEIDFEFLGNRTGQPYILQTNVFTGGKGDREQRIYLWFDPTTQYHRYSVLWNMYQIVFYVDDYPIRVFKNSNDLGVKFPFNQPMKIYNSLWNADDWATRGGLEKTDWSKAPFIASYKGFHIDGCEASVNAKFCDTQGKRWWDQPEFRDLDAAQWQKLAWVRNKYTIYNYCTDRKRYSQVPPECTRDRDI

SEQ ID NO: 3 - DNA, 861 bp, Arabidopsis thaliana
atggcgtgtttcgcaaccaaacagcctctgttgttgtctctcctccttgccattggcttctttgtggtggctgcatctgccggaaacttctatgagagctttgatatcacttggggtaatggtcgtgccaacatattcgagaatggacagcttctcacttgtactcttgacaaggtctccggctcaggttttcaatccaagaaggagtacttgtttggtaagatcgacatgaagctcaagcttgtcgctggaaactctgctggcaccgtcaccgcctactacctatcgtcaaaaggcacggcatgggatgagattgacttcgagtttttgggaaatcgcacaggacatccttacactatccacaccaatgtgttcaccggaggtaaaggcgaccgtgagatgcagttccgtctctggttcgatcccactgcggatttccacacctacaccgtccactggaaccctgttaacatcatcttccttgtggatgggatcccaattcgggtgttcaagaacaacgagaaaaatggggtggcttaccctaagaaccagccgatgaggatatactcaagcctttgggaagccgatgactgggctacagaaggcggtcgcgtgaagatcgactggagcaacgcaccattcaaggcctcttacagaaacttcaacgaccaaagctcatgcagcaggacatcaagctcaaaatgggtgacttgcgagccaaacagcaactcgtggatgtggacgactctcaatcctgcccagtacggaaaaatgatgtgggtgcaacgagacttcatgatctacaactattgtaccgattttaagagattccctcaaggcctccccaaggagtgtaaactt

SEQ ID NO: 4 - protein, 287 aa, Arabidopsis thaliana
MACFATKQPLLLSLLLAIGFFVVAASAGNFYESFDITWGNGRANIFENGQLLTCTLDKVSGSGFQSKKEYLFGKIDMKLKLVAGNSAGTVTAYYLSSKGTAWDEIDFEFLGNRTGHPYTIHTNVFTGGKGDREMQFRLWFDPTADFHTYTVHWNPVNIIFLVDGIPIRVFKNNEKNGVAYPKNQPMRIYSSLWEADDWATEGGRVKIDWSNAPFKASYRNFNDQSSCSRTSSSKWVTCEPNSNSWMWTTLNPAQYGKMMWVQRDFMIYNYCTDFKRFPQGLPKECKL

SEQ ID NO: 5 - DNA, 882 bp, Populus tremula x Populus tremuloides
atggctgctgcttatccgtggactttgtttcttggcatgctggttatggtatctggaacaatgggagctgccctgaggaagccagtggatgtggcgttcggtaggaactatgttcctacatgggcttttgaccacattaagtacttcaatggaggcaatgagattcagctgcacttggataaatacacaggtactggtttccaatcaaaaggttcatacttatttggccatttcagtatgcaaatgaagttggttcctggtgactcagctggaacagtcactgctttctatctatcctcacaaaactcggagcatgacgagatagactttgagttcttaggaaacaggactggccagccctacattttgcagacaaatgttttcacaggaggcaagggggatagagaacagaggatttacctctggtttgacccaaccaaggaattccactactattctgtcctctggaacatgtacatgatagtgttcctcgtggatgacgtgccaatcagagtgttcaagaactgcaaagatttgggagttaagtttccattcaaccagccaatgaagatctactcaagcctatggaatgccgatgattgggctaccaggggtggactcgagaagacagactggtccaaggcaccgttcattgcctcctacaggagcttccacatagatgggtgcgaggcctccgtggaagccaagttctgcgccacacagggtgctagatggtgggaccagaaggagttccaagatctggatgccttccagtacaggaggctcagctgggtccgccagaaatataccatctacaattactgcactgatagatcaagatacccttcaatgcccccagaatgcaagagagacagagacata

SEQ ID NO: 6 - protein, 294 aa, Populus tremula x Populus tremuloides
MAAAYPWTLFLGMLVMVSGTMGAALRKPVDVAFGRNYVPTWAFDHIKYFNGGNEIQLHLDKYTGTGFQSKGSYLFGHFSMQMKLVPGDSAGTVTAFYLSSQNSEHDEIDFEFLGNRTGQPYILQTNVFTGGKGDREQRIYLWFDPTKEFHYYSVLWNMYMIVFLVDDVPIRVFKNCKDLGVKFPFNQPMKIYSSLWNADDWATRGGLEKTDWSKAPFIASYRSFHIDGCEASVEAKFCATQGARWWDQKEFQDLDAFQYRRLSWVRQKYTIYNYCTDRSRYPSMPPECKRDRDI

SEQ ID NO: 7 - DNA, 891 bp, Zea mays
atggcgacggcgcggtggctgcaggttgccgccatggcggtggcgctgctggcggagtgggcgacggcggcggcgcctcggaagcccgtggacgtgccgttccagaagaactacgtgccgacgtgggcgcaggaccacatccactacatcgacggcgggcgggaggtgcagctgtacctggacaagtccaccggcacgggcttccagacgcggggctcctacctcttcggccacttcagcatgcacatgaagctcgtcggcggcgactccgcgggcaccgtcacggccttctacctgtcgtcgcagaactcggagcacgacgagatcgacttcgagttcctgggcaaccggacggggcagccctacatcctgcagaccaacgtgttcaccggcggcaagggcgaccgcgagcagcggatctacctctggttcgaccccaccaaggagtaccactcctactccgtcctctggaacctctacatgatcgcgttcttcgtggacgacgtgccgatccgggtgttcaagaacacgagcgccgacctgggcgtgcggtacccgttcagccagccgatgaagctctactccagcctgtggaacgccgacgactgggcgacccgcggcgggcgcgagaagacggactggtccaacgcgcccttcgtcgcctcctaccgcggcttccacgtcgacggctgcgaggcctccgcggaggcgcgcttctgcgccacccagggcgcgcgctggtgggaccagccggagttcagggacctcgacgccgcgcagtaccgccgcctggccgaggtcaggcgccgctacaccatctacaactactgcaccgatcgcgaccgctacggcgccgccgtgccgcccgagtgcgcccgcgaccgcgacgtc

SEQ ID NO: 8 - protein, 297 aa, Zea mays
MATARWLQVAAMAVALLAEWATAAAPRKPVDVPFQKNYVPTWAQDHIHYIDGGREVQLYLDKSTGTGFQTRGSYLFGHFSMHMKLVGGDSAGTVTAFYLSSQNSEHDEIDFEFLGNRTGQPYILQTNVFTGGKGDREQRIYLWFDPTKEYHSYSVLWNLYMIAFFVDDVPIRVFKNTSADLGVRYPFSQPMKLYSSLWNADDWATRGGREKTDWSNAPFVASYRGFHVDGCEASAEARFCATQGARWWDQPEFRDLDAAQYRRLAEVRRRYTIYNYCTDRDRYGAAVPPECARDRDV

SEQ ID NO: 9 - DNA, 882 bp, Hordeum vulgare
atgaaggctaccgcgggggccctcctcgccgtggtggccacggtgctactgcgaggcatcgcggcagcgccgccccggaagccggtggacgtgccattcgagaagaactacgtcccgacatgggcggaggaccacatccactacgtgaacggcggacgggaggtgcagctgtccctcgacaagaccaccggcactggcttccagacccggggctcctacctcttcggccacttcagcatgcacatcaagctcgtcggcggcgactccgccggcacagtcaccgccttctacctgtcgtcgcagaactcggagcacgacgagatcgacttcgagttcttggggaacaggacggggcagccgtacatcctgcagacgaacgtgttctccggcgggaagggcgaccgggaacagaggatctacctctggttcgacccaaccaaggactaccactcctactccgtcctctggaacctctacatgatcgcgttctttgtggacgacacgccgatccgggtgttcaagaacagcaaggacctcggcgtgcggtaccccttcgaccagcctatgaagctctactcgagcctgtggaacgcggacgactgggcgactcggggagggcgggagaagacggactggtccaaggcgcccttcgtcgcctcctaccggggcttccacgtcgacggctgcgaggcgtcggcggaggccaagttgtgcgccacccagggcgcccgctggtgggatcagcccgagttccaggacctggacgccgcgcagtaccgccgcctcgcctgggtcaggaaggagcacaccatctacaactactgcacagaccgcgaacgatacgccgccatgtcgcccgagtgcaaacgcgaccgcgacgtc

SEQ ID NO: 10 - protein, 294 aa, Hordeum vulgare
MKATAGALLAVVATVLLRGIAAAPPRKPVDVPFEKNYVPTWAEDHIHYVNGGREVQLSLDKTTGTGFQTRGSYLFGHFSMHIKLVGGDSAGTVTAFYLSSQNSEHDEIDFEFLGNRTGQPYILQTNVFSGGKGDREQRIYLWFDPTKDYHSYSVLWNLYMIAFFVDDTPIRVFKNSKDLGVRYPFDQPMKLYSSLWNADDWATRGGREKTDWSKAPFVASYRGFHVDGCEASAEAKLCATQGARWWDQPEFQDLDAAQYRRLAWVRKEHTIYNYCTDRERYAAMSPECKRDRDV

SEQ ID NO: 11 - DNA, 855 bp, Hordeum vulgare
atggcgtccggtcccagtagaacagtcccgtgctctgtgctgccactgctgctgctgctcgccggcgtggcccgcgcggccggcaacttctaccaggacgtggacatcacgtggggcgacgggcgcggcaagatcctcggcggcggcgacctcctcacgctgtcgctcgacagggcctccggctccgggttccagtccaagaaccagtacctgtacggccgcttcgacatgcagatcaagctcgtccccggcgactccgccggcaccgtcgccactttctacctgtcgtcgcaggggtcggcgcacgacgagatcgacttcgagttcctgggcaacgcgagcgggcagccctacacggtgcacaccaacgtgtacagccagggcaagggcggccgggagcagcagttccgcatgtggttcgaccccaccgccgacttccacacctactccgtcctctggaaccccacacacatcctgttctacgtggacgggacgccgatacgggagcaccgcaaccgggaggcggcgacgggggttccctacctgcggagccaggcgatgagggtgtacgcgagcgtgtgggacgcggaagagtgggcgacgcagggcgggcgggtgaggacggactggtcgcgggcgccgttcgtggcgtcgtacaaggggctcgccgcgagcgggtgcgcgtcgcaggacgcggcggcgtgcgccaactccaacggcgcgtggatgtaccaggagctggacgccacggcgttggaccgcctccagtgggtgcagaagaactacatgatctacaactactgcacggacacgtggaggttcaaggacggcgccccgcccgagtgcgccagcaag

SEQ ID NO: 12 - protein, 285 aa, Hordeum vulgare
MASGPSRTVPCSVLPLLLLLAGVARAAGNFYQDVDITWGDGRGKILGGGDLLTLSLDRASGSGFQSKNQYLYGRFDMQIKLVPGDSAGTVATFYLSSQGSAHDEIDFEFLGNASGQPYTVHTNVYSQGKGGREQQFRMWFDPTADFHTYSVLWNPTHILFYVDGTPIREHRNREAATGVPYLRSQAMRVYASVWDAEEWATQGGRVRTDWSRAPFVASYKGLAASGCASQDAAACANSNGAWMYQELDATALDRLQWVQKNYMIYNYCTDTWRFKDGAPPECASK

SEQ ID NO: 13 - DNA, 969 bp, Hordeum vulgare
atggcagcagccagcgctcttctcatggctgctctcgcggtcttcgccgtggccgccgccgcggcgctggacacgtcgccggtgccgttcgacgccgggtacgcgcctctcttcggcggcgacaacctcgtccggtcggcggacggccggagcgtcacgctcaagctggaccgatacaccgggtctgggttcgtgtcaaagtcggcctaccgccatggcttcttcggcgcttccattaagctgcccggcgactataccgccggcgtcgtcgtcgccttctacctatcgaactgggacgagtaccccaagaaccacgacgagctggacttcgagctgctgggcaaccggcgcggccacggctggcgtgtccagaccaacatgtacggcaacggcagcaccgcccgcggccgagaggagcgttaccatctccccgtcgaacctaccgtcaccggcgtccaccgctatgccattgcctggacccccaacaacatcgtcttctacctcgacggcgttcccatccgtgaggttgtccgcgttccctccatgggcggcgacttcccctccaagcccatgtccgtgtacgccaccatctgggacggctccgcctgggcgaccgacggcggcaagtacaaggtcgactacgcctacgcgcccttcgctgccgagttttccgacctcgtcctgagcggctgcggcgcgggcaacgtcgccgaccccgaggggtgccaggtcgacctgctcacgcacgacgtcgcagtcatggcaccggccaagcgcgccgccatgagagggttccgcgagcagcacctaacctacacggcgtgccgcgacagggtgcgatacaagaccaccgtcttccccgagtgtgacgacctcgccgacggcgactccagcttccacctctggggggagtccaagaagaggcgccgccggtcgtcctcgccgctgcagtactcttccagcatgcag

SEQ ID NO: 14 - protein, 323 aa, Hordeum vulgare
MAAASALLMAALAVFAVAAAAALDTSPVPFDAGYAPLFGGDNLVRSADGRSVTLKLDRYTGSGFVSKSAYRHGFFGASIKLPGDYTAGVVVAFYLSNWDEYPKNHDELDFELLGNRRGHGWRVQTNMYGNGSTARGREERYHLPVEPTVTGVHRYAIAWTPNNIVFYLDGVPIREVVRVPSMGGDFPSKPMSVYATIWDGSAWATDGGKYKVDYAYAPFAAEFSDLVLSGCGAGNVADPEGCQVDLLTHDVAVMAPAKRAAMRGFREQHLTYTACRDRVRYKTTVFPECDDLADGDSSFHLWGESKKRRRRSSSPLQYSSSMQ

SEQ ID NO: 15 - DNA, 897 bp, Oryza sativa
atgcgtggtggcgcctctctccggttgaggtggccggcggctctcgtcgccgtcgtagctgcggcagtgacggcggcggcggcggcggggcatggcgaccacaatttccaccgggactttgacgccgtgtgggggaagggcaatgcgaggttccgggacggcgggcggatggtggagctgacgctggacgagcagacgggggcgcggctgcagtccaaggagcggttcttgtttgggaggttcgatctcgagatcaagctcgtcaggggagagtctgcagggaccatcacttccttctatatctgcagcggcggcgcgcggcacgacgaggtggacttcgagttcctgggcaacgtgagcggcgagccgtacctgctgcacaccaacatcttcagcgacggcaagggcgagcgtgagcagcagttcgtgctctggttcgaccccaccgccgacttccacacctactccatcctctggaacccacacaacatcatactgtacattgacgggacgccgatcagggtgttcaagaacaacgaggcgtacggggtgccattcccgacgcggcagccagtgcacgtgttcgccagcatctggaacgcggaggagtgggcgacgcagggcggccgcgtcaagacggactggtcgcgcgcgccgttcgtcgccacgtaccgccgctacaacgtctccaacgcctgcgtctgggacgccgccggcgccggcgcctcccggtgcgccggcggcggcggcgggtggatgaggcggaggatggactggtggagctggatgacgctcaactgggtgcggatgaactacatggcctacgactactgcgccgacaggaagcgattcccgcaccgtttccccgccgagtgcatcatccccatcggtagaacg

SEQ ID NO: 16 - protein, 299 aa, Oryza sativa
MRGGASLRLRWPAALVAVVAAAVTAAAAAGHGDHNFHRDFDAVWGKGNARFRDGGRMVELTLDEQTGARLQSKERFLFGRFDLEIKLVRGESAGTITSFYICSGGARHDEVDFEFLGNVSGEPYLLHTNIFSDGKGEREQQFVLWFDPTADFHTYSILWNPHNIILYIDGTPIRVFKNNEAYGVPFPTRQPVHVFASIWNAEEWATQGGRVKTDWSRAPFVATYRRYNVSNACVWDAAGAGASRCAGGGGGWMRRRMDWWSWMTLNWVRMNYMAYDYCADRKRFPHRFPAECIIPIGRT

SEQ ID NO: 17 - DNA, 825 bp, Lycopersicon esculentum
atgatcaaaacatcaagttgtatatttactttctttcttctaatatgtttcttcgtagtggtggcttttggtggtactttcgaccaagaatttgatgttacatggggttatgggagggtgaaaatactcgaaaacgggcaacttcttactctttccctcgatagaagttctggctctggatttaagtctaaacaacaatatatgtttgcaaagattgacatgaagatcaaacttgtccctggtaattctgctggcaccgctactacatactatttatcttcggtggggtcagctcatgatgagattgactttgagtttcttgggaatgtaagtggagaaccatatactcttcatacaaatgtgtatgcacaaggcaagggagatagagagcaacaatttcatctttggtttgaccctactaaggatttccacacctactctattctttggaatcctcgaaatatcatatttttggtagatgggacaccaataagacaatacaagaatcttgaagcaacaaatggaataccttacccaaagaaccaaccaatgtggttatactcaagtttatggaatgctgaggaatgggcaacaagaggtggccttgtaaggactgattggagtaaagccccttttattgcttcttatagaaacttcaatgcccaaacttccaagaatcccacagccaattcttggttaactcaatcattggacaatgttgggttaacaaggatgaaatgggtgcaaaagaactatatgatatacaattattgcactgatactaaacgtttccctcaagggttccctcatgaatgcactctaaat

SEQ ID NO: 18 - protein, 275 aa, Lycopersicon esculentum
MIKTSSCIFTFFLLICFFVVVAFGGTFDQEFDVTWGYGRVKILENGQLLTLSLDRSSGSGFKSKQQYMFAKIDMKIKLVPGNSAGTATTYYLSSVGSAHDEIDFEFLGNVSGEPYTLHTNVYAQGKGDREQQFHLWFDPTKDFHTYSILWNPRNIIFLVDGTPIRQYKNLEATNGIPYPKNQPMWLYSSLWNAEEWATRGGLVRTDWSKAPFIASYRNFNAQTSKNPTANSWLTQSLDNVGLTRMKWVQKNYMIYNYCTDTKRFPQGFPHECTLN

SEQ ID NO: 19 - DNA, 990 bp, Daucus carota
atggtgaacttgtatgtgaatttgtttgtaatgtgttctgttgttgttttggctcatgggtcatcaagaaatcttccaattgtttcatttgaagaagggtactcacagctctttggtgatagtaatctcatgattcttcaagatggaaaatcagctcacctttctcttgatgaaagaacaggctctggatttgtatctcatgacctctataaacatggattctttagtgcttcaattaagttaccagcagattacacagctggggtggttgttgcattctatatgtcgaatgtagacatgtttgcaaagaaccatgatgagattgattttgagtttttggggaatattagaggcaaagagtggagacttcagaccaatgtttatggaaatggaagcacaggtgcaggcagagaagaaagatacggtctctggtttgacccttctgatgattttcatcagtacagtatattgtggagcaaagatagaattatcttttacatagacaatgttcctataagagaggtcaagaagacagaagcaatgggtggggacttcccttccaagccaatgagcttgtatgctactatttgggatggatctaattgggctacaaatggaggtaaatacaaggtcaattataaatattccccttacattgccgagttctccgatttcgtgcttcacggttgtgccgttgatcccatcgagatgtcgacctcatgtgatactgcgcccaagtctcaatctgttcctacaggtacaacaaaagaaagtagaactaaaatgcagaacctgaggaaaaaatacatgcagtactcctattgttatgatacaactcgataccaggttccaccttctgagtgtgtaatcgatccacttgagtccgagagacttagaggattcgacccggtaacatttggtacaagccaccgcgggcatggtaaaagacatcacaaccgccgatcataccgtcatggaataaactctgtt

SEQ ID NO: 20 - protein, 330 aa, Daucus carota
MVNLYVNLFVMCSVVVLAHGSSRNLPIVSFEEGYSQLFGDSNLMILQDGKSAHLSLDERTGSGFVSHDLYKHGFFSASIKLPADYTAGVVVAFYMSNVDMFAKNHDEIDFEFLGNIRGKEWRLQTNVYGNGSTGAGREERYGLWFDPSDDFHQYSILWSKDRIIFYIDNVPIREVKKTEAMGGDFPSKPMSLYATIWDGSNWATNGGKYKVNYKYSPYIAEFSDFVLHGCAVDPIEMSTSCDTAPKSQSVPTGTTKESRTKMQNLRKKYMQYSYCYDTTRYQVPPSECVIDPLESERLRGFDPVTFGTSHRGHGKRHHNRRSYRHGINSV

SEQ ID NO: 21 - DNA, 891 bp, Carica papaya
atggcaaagcttagttgtgtgttaatggctccactactattctcttttctgctcattctcatggtttcttctcagggtcctccctcaccagggtactatcctacatccagattcccgtctttaggttttgatcaaggattcagaaatcgttggggtcctcagcaccagagagtagaacagggtgtcctaacaatctggcttgattctacctcaggaagtgggttcaaatcacttcatacatatcaatctggttattttggagctgccattaaacttcaacccggttatactgctggagtaattacatctttctatctttcaaataatgaagattatccaggaaatcatgatgaaattgacattgaatttctgggtacaactccaggggaaccctacactttgcagacaaatgttttcataagaggaagtggagatagaaatatcgtcggcagggaagtcaaatttcatctctggtttgatccaacacaagacttccataactatgctattctctggaccccaagtgagatcgtattttttgtggatgatgtgcctattcggaggtatccaaggaagagtgatgccacatttcccttaagaaccatgtgggtttatgggtcaatttgggatgcttcatcttgggctactgacgatggaaaatacaaagctgactaccaataccaaccctttgttggtaggtataagaatttcaaaatagccgcgtgcagagccgacgggcaagcttcctgtaggcccccttctgtgtctccatctggctttggcgtgttgagcccacaacaagaatcggccatggaatgggcacagagaaattctttggtttataactattgtttagaccctaagagagatcataccccaacgcccgagtgt

SEQ ID NO: 22 - protein, 297 aa, Carica papaya
MAKLSCVLMAPLLFSFLLILMVSSQGPPSPGYYPTSRFPSLGFDQGFRNRWGPQHQRVEQGVLTIWLDSTSGSGFKSLHTYQSGYFGAAIKLQPGYTAGVITSFYLSNNEDYPGNHDEIDIEFLGTTPGEPYTLQTNVFIRGSGDRNIVGREVKFHLWFDPTQDFHNYAILWTPSEIVFFVDDVPIRRYPRKSDATFPLRTMWVYGSIWDASSWATDDGKYKADYQYQPFVGRYKNFKIAACRADGQASCRPPSVSPSGFGVLSPQQESAMEWAQRNSLVYNYCLDPKRDHTPTPEC

SEQ ID NO: 23 - DNA, 864 bp, Festuca pratensis
atggggaagcctgcagcgttgattccggtgctagctcttcttgcctgcttggcttttgggcttgtgtccggcggcaacttcaacgacgattgcgacgcgacctgggagccccagaactgctgggtttatgatgctggcaatagcctctccctcgccctcgtcagcaactcctcaggctcgatgatccggtccaagaggcagttcatctttgggaccgtctccacatggatccaagcttcgtcaagggcaactcttgcccggcaccgtcacaacatattacctatcctcggcggagacaatcatgacgaaatcgacttcgagttcctggggaacgagaccggacagccctacaccctgcacaccaacgtctacgccgacggcgtcggcaacaaggagatgcagttcgtgccctggttcgaccccaccggcggttaccacaagtacaccatctcctggacgccctgcatgatcgtctggtacgtcgacgacctccccatccgggtcttccgcaattaccgccagagcaacggcgtggccttcccgacgagccgccccatgtacgcctactccagcatctggtccgccgacgactgggccacgcagggcggccgcgtcaagaccgactggacccacgcgcccttcgtcgccaactaccacggcatcgacctcgacatctgcgagtgctacggcggcgactgcatctacggctgcgccggcgcctacggccagccctactgcggcgggctcaccgacgagcagcgctggaagatgcagtgggtacaggagagatacaggatctacgactactgcgtggactacaaggacggcaaggtgcccggcgtcgagtgcagcctgccccagtac

SEQ ID NO: 24 - protein, 288 aa, Festuca pratensis
MGKPAALIPVLALLACLAFGLVSGGNFNDDCDATWEPQNCWVYDAGNSLSLALVSNSSGSMIRSKRQFIFGTVSTWIQASSRATLARHRHNILPILGGDNHDEIDFEFLGNETGQPYTLHTNVYADGVGNKEMQFVPWFDPTGGYHKYTISWTPCMIVWYVDDLPIRVFRNYRQSNGVAFPTSRPMYAYSSIWSADDWATQGGRVKTDWTHAPFVANYHGIDLDICECYGGDCIYGCAGAYGQPYCGGLTDEQRWKMQWVQERYRIYDYCVDYKDGKVPGVECSLPQY

SEQ ID NO: 25 - DNA, 933 bp, Oryza sativa
atggcgaaggctctcctcgccgtcgtcgtcgtcgccgtggcggcggtgctcgagctggggctcgtcggagccaacttccaggatcagtgtgacatcacgtgggagccacagaacgccaagatgacggagggaggagaccacctcacgctctcccttgtcagcaactcctctggttgtatgctccggaccaagaagcagttcatctacggcagcgtgtcgactcgcatccagcttgtgaagggcaactcggccggcaccgtcaccacctactacacatcgtccatcggcgacaaacacgacgagatcgacttcgagttcctgggcaactccagcggcctcccctacaccttccacaccaacgtcttcgccgacggcgtcggcagccgcgagatgcagttccgcccctggttcgaccccaccgacggctaccacaactacaccatcttctggaacccctgcatgatcgtgtggttcgtggacagcatcccaatcagggtgttccggaaccacgagaaggaaggggtgccgttcccgacaaagcggccgatgtacgccttctccagcatctgggcggcggaggattgggccacgcagggtggccgcgtcaagacggactggaccaaggcccctttcgtcgccgagtaccgtgacatcggcctcaacatctgtgagtgccccggctcaggctctggctccagctccagcttcagctccagctccagctctacctccggcgatgccgaggacccagcgtgcgcgcagcggtgcgcgacgtcggaccactggtacgcggcggaggggctgtgccagctgagcgacaagcagctgcggcagatgaaggcggtgcagctgggctacaccatctacgactactgcgccgacgcccaggccaagggacgccccgtgccgccggagtgcagcatgccgcagtac

SEQ ID NO: 26 - protein, 311 aa, Oryza sativa
MAKALLAVVVVAVAAVLELGLVGANFQDQCDITWEPQNAKMTEGGDHLTLSLVSNSSGCMLRTKKQFIYGSVSTRIQLVKGNSAGTVTTYYTSSIGDKHDEIDFEFLGNSSGLPYTFHTNVFADGVGSREMQFRPWFDPTDGYHNYTIFWNPCMIVWFVDSIPIRVFRNHEKEGVPFPTKRPMYAFSSIWAAEDWATQGGRVKTDWTKAPFVAEYRDIGLNICECPGSGSGSSSSFSSSSSSTSGDAEDPACAQRCATSDHWYAAEGLCQLSDKQLRQMKAVQLGYTIYDYCADAQAKGRPVPPECSMPQY

SEQ ID NO: 27 - DNA, 873 bp, Sorghum bicolor
atggcgcggccggcggcgtggactccgcctgctctgtcgctcgcggtcgccgtcctctgcctcgtcggccggtccacctccgcggagttctggctcgacgagttcaccacggacggcgacgtccgcaaagactacgacggttctggccggcaggtggcgtcgctggtcctcgaccagagttctggcgctggcttcaactccacccgcaagtacctcttcggcgagttcagcgtcgagatgaagctcgtcgccggcaactccgccggcactgtcacctccttctacctgacgtccggcgagggcgacgagcacgacgagatcgacatggagttcatgggcaactccagcgggtcgccgacggtgctcaacaccaacgtgtgggccagcggcgacggcaagaaggagcaccagttctacctgtggttcgacccggccgccgacttccacaagtacaagatcacctggaacgacaagaacatcatcttccaggtggacgacgtgacggtgcgcgtcttcaagcgctacgccgacctgccgtacccgagctccaagcccatggcggtgcacgccacgctgtgggacggcagctactgggccaccgagaagggcaaggtgcccatcgactggtcccgcgcgcccttcgtcgtctcctaccgagcatacacggccaacgcctgcgccgtcggcggcggcggcgggggctcgtcgtcgtcgtgccccgacgccgccggtgggaacgagtggatggacaggcagcccgacgacaccgaccggctcaccgtggagtgggcgcgccggaactgcttgcagtacaactactgcgaggacgggtggcgcttcccgcaggggttccccggcgagtgcgcccgcaac

SEQ ID NO: 28 - protein, 291 aa, Sorghum bicolor
MARPAAWTPPALSLAVAVLCLVGRSTSAEFWLDEFTTDGDVRKDYDGSGRQVASLVLDQSSGAGFNSTRKYLFGEFSVEMKLVAGNSAGTVTSFYLTSGEGDEHDEIDMEFMGNSSGSPTVLNTNVWASGDGKKEHQFYLWFDPAADFHKYKITWNDKNIIFQVDDVTVRVFKRYADLPYPSSKPMAVHATLWDGSYWATEKGKVPIDWSRAPFVVSYRAYTANACAVGGGGGGSSSSCPDAAGGNEWMDRQPDDTDRLTVEWARRNCLQYNYCEDGWRFPQGFPGECARN

SEQ ID NO: 29 - DNA, 840 bp, Sorghum bicolor
atggcgcggcggtctctggcccttctgctcgcgtcgtcgctggccctcgtgatgatggcggccgtcgcgtcggcggactcgtggctctacgagaagttcagcacggacggcaccgtccgcaccaactacgacgagtccggcgcgcaggtggcgatgctcaacctcgaccgcagctccggcgccggcttcaactccaaggagcagtacctctacggccagttcagcgtccagatgaagctcatccccggcaactccgccggcaccgtctcctgcttctacctttcttctggcgacggcgacgggcatgacgagatcgacatggagttcatgggcaacgccacggggcagccggtggtgctcaacacaaacgtgtgggccaacggcgacggcaagaaggagcaccagttcgacctgtggttcgacccggccgccgactaccacacctacaccatcatctggaacccgaccaacatcctcttcaaggtggacgacaacgtgatccgcgtcttcaagcgctacgccgacctggcctacccgagctccaagcccatgacgctgcacgccacgctgtgggacggcagctactgggccaccgagaagggcaaggtgcccatcgactggtccggcgcgcccttcgtcgtctcctacaaaggctactccgccgacgcctgcgtcagcggcggcgcctgcgccgccggcagcggcgcgtggatgaacaagcagcctgacagcgccgagtggggcaccgtcaagtgggccgagagcaactacatgcgctacaactactgcgacgacggctggaggttcccgcaggggctgccggcggagtgcagccgcagc

SEQ ID NO: 30 - protein, 280 aa, Sorghum bicolor
MARRSLALLLASSLALVMMAAVASADSWLYEKFSTDGTVRTNYDESGAQVAMLNLDRSSGAGFNSKEQYLYGQFSVQMKLIPGNSAGTVSCFYLSSGDGDGHDEIDMEFMGNATGQPVVLNTNVWANGDGKKEHQFDLWFDPAADYHTYTIIWNPTNILFKVDDNVIRVFKRYADLAYPSSKPMTLHATLWDGSYWATEKGKVPIDWSGAPFVVSYKGYSADACVSGGACAAGSGAWMNKQPDSAEWGTVKWAESNYMRYNYCDDGWRFPQGLPAECSRS

SEQ ID NO: 31 - DNA, 870 bp, Arabidopsis thaliana
atggactatatgagaatctttagtgtttttgtagtaacgttatggatcataagggttgatgctagggttttcggaggacgaggaatcgaaaaatttgtaactttcggtcagaattatattgttacgtggggacaaagtcatgtttctacactccattctggcgaagaagttgatctttacatggatcaatcttcaggtggtgggttcgaatccaaggacgcctacggatcaggtctctttgaaatgagaatcaaagtgccttcaggaaatactggcggcatcgtcaccgctttttacttgacgtcgaaaggaggtggtcacgacgagattgacttcgagtttctagggaacaacaacggaaaaccggtaacgttacagacaaatctgtttctaaatggagaaggaaacagagaagagaggtttttgctttggttcaatccaaccaaacactaccacacttatgggcttctttggaacccttaccaaattgtgttttacgtggacaacatcccaataagagtatacaaaaacgaaaacggcgtaagttatccatcaaagcctatgcaagtcgaggctagtctctggaacggtgacgattgggcgactgatggtggtcggactaaggttaactggtcatactctcctttcattgcacatttccgagacttcgccttatccggctgcaacatagatggtcggagtaataatgtcggcgcttgtgaatcctccaactactggtggaatgcaggcaactatcaaagattaagcggaaacgaacagaaactttatgaacatgtgaggagcaagtacatgaactatgactattgtactgatcgctctaagtaccaaactcctcctagagaatgttat

SEQ ID NO: 32 - protein, 290 aa, Arabidopsis thaliana
MDYMRIFSVFVVTLWIIRVDARVFGGRGIEKFVTFGQNYIVTWGQSHVSTLHSGEEVDLYMDQSSGGGFESKDAYGSGLFEMRIKVPSGNTGGIVTAFYLTSKGGGHDEIDFEFLGNNNGKPVTLQTNLFLNGEGNREERFLLWFNPTKHYHTYGLLWNPYQIVFYVDNIPIRVYKNENGVSYPSKPMQVEASLWNGDDWATDGGRTKVNWSYSPFIAHFRDFALSGCNIDGRSNNVGACESSNYWWNAGNYQRLSGNEQKLYEHVRSKYMNYDYCTDRSKYQTPPRECY

SEQ ID NO: 33 - DNA, 906 bp, Glycine max
atggatgcaagggcatcatcttcttcaatgtccatgattcttttcattacagctattgtggctgcagcttattttcattcatcagaagcagctgtgtcaaaaggatcttttgaggacaatttcagcataatgtggtctgaggaccattttactacctctaaagatggacagatctggtatctctcactagacaaagacacaggatgtgggtttcaaacaaaacaacgctacagattcgggtggttcagtatgaagctgaagttggtagcaggtgactctgcaggtgttgtgacagcttattatatgtgctcagaaaatggtgcagggccggagagggatgagcttgattttgagtttttgggaaacagaactggggagccttatttgattcagactaatgtgtacaagaatgggactggagggcgtgagatgaggcatatgctatggtttgaccccacagaggactaccacacttattccattctctggaacaaccaccagatagtgttttttgtggatagagttccagtgagggtgttcaagaacaatggggaaccaaacaacttcttcccgaatgagaagcccatgtacttgttctccagcatatggaatgcagatgagtgggccacaagaggtgggcttgagaagacaaactggaaattagccccatttgtgtcatcctacaaagacttcagtgtggatggttgccagtgggaagacccataccctgcctgtgtctcaaccacaaccaaaaattggtgggatcagtatgatgcttggcacctttctgatgatcagaaaaaggattatgcttgggttcagaggaaccttgttatctatgactactgccaggattctgcacgttatccaactaccccagaggagtgttcattgagtccatgggat

SEQ ID NO: 34 - protein, 302 aa, Glycine max
MDARASSSSMSMILFITAIVAAAYFHSSEAAVSKGSFEDNFSIMWSEDHFTTSKDGQIWYLSLDKDTGCGFQTKQRYRFGWFSMKLKLVAGDSAGVVTAYYMCSENGAGPERDELDFEFLGNRTGEPYLIQTNVYKNGTGGREMRHMLWFDPTEDYHTYSILWNNHQIVFFVDRVPVRVFKNNGEPNNFFPNEKPMYLFSSIWNADEWATRGGLEKTNWKLAPFVSSYKDFSVDGCQWEDPYPACVSTTTKNWWDQYDAWHLSDDQKKDYAWVQRNLVIYDYCQDSARYPTTPEECSLSPWD

SEQ ID NO: 35 - DNA, 873 bp, Medicago truncatula
atggccatattttacccctttaaaaacaatggtgtcttttttatgttgttattgtggattgtcgtatcatctgtttggggaagaccagcaacttttaatcaagattttcatgtcacgtggtcagaaccccatatcaagcaaattgatcaaggcagaactatccaacttaccctagaccaaggctctggttgcgggtttgcatcaaaggtgaagtatttgtttggtcgtgtaagcatgaagatcaaacttgtccctggagattctgctggtactgttactgcattttatatgaactctgacactgacagtgtacgtgatgagttggattttgagttcttgggaaaccgtactggacaaccttacacagttcaaacaaacatttatgctcatggaaagggtgatagagaacaaagagttaatctttggtttgatccttctgctgatttccacacttactccattctttggaatcatcaccatattgtgttctacgttgatgaagttcccattagagtatacaagaacaatgaagcaaaagggataccataccccaaaatgcaagccatgggagtattttcaacattatgggaagctgataattgggcaacaagaggtggattagagaaaattaattggagtaaagcacctttttatgcttattacaaggattttgatattgaaggatgtgcaatttcaggacccaatacatgttccactaaccccaaaaattggtggggaggggttgaatatcaagcttttagtgccattgaagctagaaggtataggtgggtttgtatgaaccatgtgatctatgattattgtcaagataaatcaaggtacccaatgaccccacatgagtgtctttcaggcatt

SEQ ID NO: 36 - protein, 291 aa, Medicago truncatula
MAIFYPFKNNGVFFMLLLWIVVSSVWGRPATFNQDFHVTWSEPHIKQIDQGRTIQLTLDQGSGCGFASKVKYLFGRVSMKIKLVPGDSAGTVTAFYMNSDTDSVRDELDFEFLGNRTGQPYTVQTNIYAHGKGDREQRVNLWFDPSADFHTYSILWNHHHIVFYVDEVPIRVYKNNEAKGIPYPKMQAMGVFSTLWEADNWATRGGLEKINWSKAPFYAYYKDFDIEGCAISGPNTCSTNPKNWWGGVEYQAFSAIEARRYRWVCMNHVIYDYCQDKSRYPMTPHECLSGI

SEQ ID NO: 37 - DNA, 897 bp, Arabidopsis thaliana
atgactctaataaatcgctcaaaaccatttgtacttctcgtaggcttctcaattatctcaagtttgctattatgggtctcacaagcttccgttgtttcatcaggagattttaacaaagacttctttgtgacatggtctcctacccacgttaacacctccaatgatggtcgatcaagaacccttaaactcgatcaagaatctggagcttctttttcttccattcagacgttcttgttcggccaaatagatatgaagatcaaacttatccgtggaagttctcaaggaaccgtggttgcttattatatgtcctcagatcagcctaaccgggatgagattgactttgagttcctcggaaacgttaatgggcagccttacattctccagacaaatgtctatgccgagggacttgataaccgcgaagaaaggatccatctctggttcgacccagccaaggactttcacacttactctatcttgtggaacattcaccaaattgtatttatggtggatcagattccgataaggttgtatagaaaccatggggagaaaggcgtagcctacccaaggttgcaacctatgagtgtacaggcgagtctatggaacggtgagagctgggctacgcgcggtggacatgacaagattgattggtcaaagggtccatttgtggcttcctttggggattacaagatagacgcttgtatttggataggcaatacaagtttttgtaatggagagagcactgaaaactggtggaacaagaatgagttcagttctttgacaagagtgcaaaagagatggtttaaatgggtgagaaaatatcacttgatttatgattattgccaagattatggaaggttcaataacaagctaccgaaggaatgttctcttcctaaatat

SEQ ID NO: 38 - protein, 299 aa, Arabidopsis thaliana
MTLINRSKPFVLLVGFSIISSLLLWVSQASVVSSGDFNKDFFVTWSPTHVNTSNDGRSRTLKLDQESGASFSSIQTFLFGQIDMKIKLIRGSSQGTVVAYYMSSDQPNRDEIDFEFLGNVNGQPYILQTNVYAEGLDNREERIHLWFDPAKDFHTYSILWNIHQIVFMVDQIPIRLYRNHGEKGVAYPRLQPMSVQASLWNGESWATRGGHDKIDWSKGPFVASFGDYKIDACIWIGNTSFCNGESTENWWNKNEFSSLTRVQKRWFKWVRKYHLIYDYCQDYGRFNNKLPKECSLPKY

SEQ ID NO: 39 - DNA, 870 bp, Arabidopsis thaliana
atggtcggtatggatttgttcaaatgtgtaatgatgataatggtgttggttgtctcttgtggtgaagctgtttctggagctaagttcgatgagctttatcggtcaagctgggctatggatcattgtgtcaacgaaggcgaagtcacaaaactcaagcttgacaattactctggagctgggtttgaatcaagaagcaaatacttgtttggtaaagtctctatccagattaagcttgttgagggtgattcagcaggaacagtcactgctttctacatgtcttcagatggtccgaaccacaacgaattcgattttgagttcttagggaacactaccggcgagccttacatagtccagaccaatatctacgtgaacggagttggaaacagagaacaaagactcaacctttggttcgatcccaccactgagtttcacacttactcaatcctctggagtaaacgcagtgttgtattcatggtagacgaaacaccgattcgagttcagaagaatcttgaagagaaaggtatcccatttgctaaagatcaagcaatgggtgtttacagctccatttggaatgcagatgattgggctacacaaggaggtcttgttaaaactgattggagtcacgctcctttcgttgcttcttacaaagaattccaaatcgatgcatgtgagattccaacaactactgatctaagcaagtgtaatggagaccagaagttttggtgggatgaaccaactgtctctgagcttagccttcatcagaatcatcagcttatttgggttcgagctaatcatatgatttatgattattgttttgatgctactaggtttcctgttactcctcttgagtgccaacatcatcgtcatttg

SEQ ID NO: 40 - protein, 290 aa, Arabidopsis thaliana
MVGMDLFKCVMMIMVLVVSCGEAVSGAKFDELYRSSWAMDHCVNEGEVTKLKLDNYSGAGFESRSKYLFGKVSIQIKLVEGDSAGTVTAFYMSSDGPNHNEFDFEFLGNTTGEPYIVQTNIYVNGVGNREQRLNLWFDPTTEFHTYSILWSKRSVVFMVDETPIRVQKNLEEKGIPFAKDQAMGVYSSIWNADDWATQGGLVKTDWSHAPFVASYKEFQIDACEIPTTTDLSKCNGDQKFWWDEPTVSELSLHQNHQLIWVRANHMIYDYCFDATRFPVTPLECQHHRHL

SEQ ID NO: 41 - DNA, 912 bp, Sagittaria pygmaea
atggctctccgaccgtctttacataaacagttcgctctcctagtggcagcgtctgtgctggccttttgctgctcggcagcaggccgcagcgtcggcctcgccccggcctcgccctccagcttcaacaacgatttcgacatcacctggggtgatggccgggggaagatcctaaacaacggccagctcctcagcctctccctggacaaaacctccggctcagggtttcaatctaaaaaccagtacctctttggcaacatcgacatgcaactgaagctcgtccccggcaactcggctggaacggtcaccgcctattacctatcctcacagggccctacccacgacgagatcgactttgagttcctcgggaacgtctccggccagccctacaccctgcacaccaacgttttctcccaggggaaaggcaacagggagcaacagttccggctttggttcgaccccaccaaggacttccacacctattcgatcctctggaaccccagccacatcgtcttctacgtcgacggcacccccatcagagacttccgcaataacgaggccagaggagttcccttccccaaggcccagccgatgcggatctattccagtctttggaacgccgacgactgggctacccaaggaggccggatcaagacagactggacgcaggcgccattcacggcgtcctacaggggattcaaggcggacgcatgtgccgttggatcaactggatcctcctccaaatgcgccgccttgcagaccaccgccggcaacaccaactggtggatccggcaggagctcgatgccaccggccaggagcgcatgcgctgggtgcagcgcaaccacatgatctacaactactgcacagacgtcaaccgcttccctcaggggttacccactgagtgctccgttgcc

SEQ ID NO: 42 - protein, 304 aa, Sagittaria pygmaea
MALRPSLHKQFALLVAASVLAFCCSAAGRSVGLAPASPSSFNNDFDITWGDGRGKILNNGQLLSLSLDKTSGSGFQSKNQYLFGNIDMQLKLVPGNSAGTVTAYYLSSQGPTHDEIDFEFLGNVSGQPYTLHTNVFSQGKGNREQQFRLWFDPTKDFHTYSILWNPSHIVFYVDGTPIRDFRNNEARGVPFPKAQPMRIYSSLWNADDWATQGGRIKTDWTQAPFTASYRGFKADACAVGSTGSSSKCAALQTTAGNTNWWIRQELDATGQERMRWVQRNHMIYNYCTDVNRFPQGLPTECSVA

SEQ ID NO: 43 - DNA, 876 bp, Hordeum vulgare
atggctcgcatgggggcgtcggtgctggtgatcctgctcgcctcttgtgccctggcggcggcgagcttcgacaaggagttcgacgttacctggggtgacgggcgcggcaagatcctcaacaatggccagctgctgatgctggggctggacaaggtctccggctccgggttccagtccaagcgcgagtacctcttcggcaagatcgacatgcagctcaagctcgtccccggcaactccgccggcaccgtcaccgcatattacctgtcgtcgcagggtccgacgcacgacgagatcgacttcgagttcctgggcaacgtcaccggcgagccatacacgctgcacaccaacgtgttcacgcaggggcagggccagcgggagcagcagttccgcctctggttcgatcctaccaacgacttccacacctactccatcctctggaacccgaagcacatcatcttcttggttgacgacatgccgatcagggacttcaggaacatggagggaaaggggatcgccttccccaagaaccagcctatgcggctgtactccagcctctggaacgccgacgactgggcgacacagggtggccgcgtcaagaccgactggtcccacgctccgttttccgcctcctaccgcggcttcaaggccgacgcgtgcgtggtgaccgtaggtggccggccgcgctgcggcgccagcatcggcacggacgcggccccagggaccggcggcgcggccgcggtcggcgactggtacaaccaagagctggatcttacgcggcagcagcgcatgcgttgggtgcagagcaattacatgatctacaactactgcactgaccccaagcgcgtcgccaagggcgtccctgccgagtgctccatg

SEQ ID NO: 44 - protein, 292 aa, Hordeum vulgare
MARMGASVLVILLASCALAAASFDKEFDVTWGDGRGKILNNGQLLMLGLDKVSGSGFQSKREYLFGKIDMQLKLVPGNSAGTVTAYYLSSQGPTHDEIDFEFLGNVTGEPYTLHTNVFTQGQGQREQQFRLWFDPTNDFHTYSILWNPKHIIFLVDDMPIRDFRNMEGKGIAFPKNQPMRLYSSLWNADDWATQGGRVKTDWSHAPFSASYRGFKADACVVTVGGRPRCGASIGTDAAPGTGGAAAVGDWYNQELDLTRQQRMRWVQSNYMIYNYCTDPKRVAKGVPAECSM

SEQ ID NO: 45 - DNA, 849 bp, Glycine max
atggctcctagttctgctcacaacaatgggttctatgtgcttatgctagttgggatagtggttagcactatggttgctacctgtgctggtagcttctaccaagactttgatctaacatggggtggtgaccgtgctaagatattcaatggtggccagcttctatcactttccctagacaaagtctctggctctggcttcaaatcaaagaaagaatacctatttgggaggattgatatgcagctcaagctcgttgccggcaactctgctggcactgtcactgcttactacttgtcatcccaagggccaacacatgatgagattgatttcgagtttttgggaaacctaagtggggacccttatattctccacacaaacatcttcacccaaggcaaaggcaacagggagcaacagttctatctctggttcgaccccaccagaaacttccacacttactctatcatttggaagccccagcacatcatattcttggttgataacacacccataagggtattcaagaatgctgaacctcttggtgttccttttccaaagaaccagcccatgagaatctattctagcctctggaatgctgatgactgggccaccagaggaggattggtgaaaactgattggtccaaagcaccctttacagcatactaccgcaatttcaaggccattgagttctcatccaagtcttccatttcaaattctggggctgaatatgaggcaaatgagcttgatgcttatagcagaagaagactgagatgggttcagaagtacttcatgatctataactactgcagtgatctcaagcgattcccacaaggtcttcctgctgaatgtaaacgt

SEQ ID NO: 46 - protein, 283 aa, Glycine max
MAPSSAHNNGFYVLMLVGIVVSTMVATCAGSFYQDFDLTWGGDRAKIFNGGQLLSLSLDKVSGSGFKSKKEYLFGRIDMQLKLVAGNSAGTVTAYYLSSQGPTHDEIDFEFLGNLSGDPYILHTNIFTQGKGNREQQFYLWFDPTRNFHTYSIIWKPQHIIFLVDNTPIRVFKNAEPLGVPFPKNQPMRIYSSLWNADDWATRGGLVKTDWSKAPFTAYYRNFKAIEFSSKSSISNSGAEYEANELDAYSRRRLRWVQKYFMIYNYCSDLKRFPQGLPAECKR

SEQ ID NO: 47 - DNA, 849 bp, Cucumis sativus
atggcttccacaactctgtttctcacgcttcttttctctgcttccctaattgccatctcttcggctaacttcaaccaagattttcaaatcacttggggagatggtcgagcaaagattctcaacaatggtgaacttctaactctgtcccttgacaaggcctctggctctggcttccagtctcaaaacgagtacttgtttggaaagattgatatgcaactcaagcttgtggctggcaattctgctggcactgtcactgcttattatttgtcttcgaaaggatctacatgggatgagattgactttgagttcttggggaatttgagtggtgacccttacactctccataccaatgtgtttagccaagggaaaggcaacagagaacaacagttccatctttggtttgaccccactgctgatttccacacctattccattctttggaaccccaatcgtattatattttcggtggacggtactccgataagagaattcaagaactgggaatcgaacggcgtgccatttccgaaggaccagccaatgagaatatactcgagcttatggaacgcggacgattgggcaacaagaggagggctggtgaagacggactggacaaaggctcctttcactgcttcctacaaaaacttcaaagccgaagcctgcgtttggtccggggggaaatcttcttgcagcccctcgccggcggggtcgagcaactcatggctgtcccaagagatggactctgcaagtcaagagagactaaaatgggtgcaaaagaattatatgatttacaactattgtacggacaccaagaggtttcctcaaggcctccctacagagtgcaacttgcca

SEQ ID NO: 48 - protein, 283 aa, Cucumis sativus
MASTTLFLTLLFSASLIAISSANFNQDFQITWGDGRAKILNNGELLTLSLDKASGSGFQSQNEYLFGKIDMQLKLVAGNSAGTVTAYYLSSKGSTWDEIDFEFLGNLSGDPYTLHTNVFSQGKGNREQQFHLWFDPTADFHTYSILWNPNRIIFSVDGTPIREFKNWESNGVPFPKDQPMRIYSSLWNADDWATRGGLVKTDWTKAPFTASYKNFKAEACVWSGGKSSCSPSPAGSSNSWLSQEMDSASQERLKWVQKNYMIYNYCTDTKRFPQGLPTECNLP

SEQ ID NO: 49 - DNA, 870 bp, Oryza sativa
atggggcaacctagggctcaactcctaccctccatgtccatggcagctttgtatctcatcctagccacctccccagtaatcagcgacatgaccgacagccttgacatgctgtggggcaatacacaggtgctctatgacagcactggccaccaaattgtatcactgtcccttgaccgctggactacttctgcattccgctcaaagactaagtatctctttgcgaggattgacatggacatcaagcttgttgccaaggactcagccggcactgtcaccacattatatatgataactgaggggctgtgggacatacatgatgagattgatctcgagttcttggggaacaccaccggtgagccctacaccttgcacactaacatctatgccagaggaacaggtggccgagaaaagcagtaccggctttggtttgatccaacagaagacttccatacctacaccatcatctggaacccgcagatgatcttaatacttgttgatggcacaccaattcggcagatgaagaatcagctaaggaatgatatacccttcccgctatatcaaccaatgaggctgtacgccagcatctgggatgccgatgactgggcaacgcagggtgggcgtatcaaaaccgactggtctcaagcaccatttaccgcattcttccggaactaccaagctaacgcctgcattccgtacaaaactgcctggatttgcagccaagggtctaatgacagcagctggtttacccaggatttggatgaggaagggaagcagaaacttaaggacgtagatgataactacaagatttatgattactgcactgactcaaggaggtacccaaatgggtatcccccagagtgcgggtcacag

SEQ ID NO: 50 - protein, 290 aa, Oryza sativa
MGQPRAQLLPSMSMAALYLILATSPVISDMTDSLDMLWGNTQVLYDSTGHQIVSLSLDRWTTSAFRSKTKYLFARIDMDIKLVAKDSAGTVTTLYMITEGLWDIHDEIDLEFLGNTTGEPYTLHTNIYARGTGGREKQYRLWFDPTEDFHTYTIIWNPQMILILVDGTPIRQMKNQLRNDIPFPLYQPMRLYASIWDADDWATQGGRIKTDWSQAPFTAFFRNYQANACIPYKTAWICSQGSNDSSWFTQDLDEEGKQKLKDVDDNYKIYDYCTDSRRYPNGYPPECGSQ

SEQ ID NO: 51 - DNA, 876 bp, Artificial Sequence
  Artificial DNA Sequence
atgggctcgtccctctggacttgtttgatcctcctctccttggcatcggcatccttcgcagcgaaccctcgaactccgatcgatgtgcctttcggacggaactacgtgccgacatgggcattcgaccacattaagtatttgaacggaggctcggagatccagttgcatctcgacaagtacaccggcactggtttccagtcgaagggctcctacttgttcggacatttctccatgtacatcaaattggtgcctggtgactcggcaggaactgtcaccgcattctacctctcgtcgacaaacgcagagcatgacgaaatcgacttcgagttcctcggcaacaggacaggacagccgtacatcctccagaccaacgtcttcacaggaggcaaaggtgatcgggaacagcggatctacttgtggttcgatcccacaacccagtaccataggtactcggtgctctggaacatgtatcagatcgtcttctacgtcgacgattatccgatccgagtgttcaagaactccaacgacttgggcgtcaaattccccttcaaccagcccatgaagatttacaactcgttgtggaacgccgacgattgggcaaccaggggtggtctcgagaagacagattggtcgaaagcacctttcatcgcgtcgtacaagggtttccacatcgacggatgtgaagcctccgtgaacgccaagttctgtgacacccagggcaaacgatggtgggatcagccggaattccgggatttggatgcagcccagtggcagaagctcgcgtgggtcaggaacaagtacaccatctataactactgtaccgatcggaaacgatattcgcaggtgcctcccgagtgtacacgcgatagggacatc

SEQ ID NO: 52 - DNA, 36 bp, Artificial Sequence
  Artificial DNA Primer
acatgtctttgataagctagcgggccgcatcatgta

SEQ ID NO: 53 - DNA, 36 bp, Artificial Sequence
  Artificial DNA Primer
tacatgatgcggcccgctagcttatcaaagacatgt

SEQ ID NO: 54 - DNA, 41 bp, Artificial Sequence
  Artificial DNA Primer
ttaatcgccttgcagcacaccgcttcctcgctcactgactc

SEQ ID NO: 55 - DNA, 47 bp, Artificial Sequence
  Artificial DNA Primer
acaataaccctgataaatgcggaacaacactcaaccctatctcggtc

SEQ ID NO: 56 - DNA, 53 bp, Artificial Sequence
  Artificial DNA Primer
agatagggttgagtgttgttccgcatttatcagggttattgtctcatgagcgg

SEQ ID NO: 57 - DNA, 42 bp, Artificial Sequence
  Artificial DNA Primer
ttctacacgaaggaaagaggaggagagagttgaacctggacg

SEQ ID NO: 58 - DNA, 47 bp, Artificial Sequence
  Artificial DNA Primer
aggttcaactctctcctcctctttccttcgtgtagaagaccagacag

SEQ ID NO: 59 - DNA, 43 bp, Artificial Sequence
  Artificial DNA Primer
tcagtgagcgaggaagcggtgtgctgcaaggcgattaagttgg

SEQ ID NO: 60 - DNA, 50 bp, Artificial Sequence
  Artificial DNA Primer
ttcctcaatcctctatatacacaactggccatgggctcgtccctctggac

SEQ ID NO: 61 - DNA, 45 bp, Artificial Sequence
  Artificial DNA Primer
agctcgctagagtcgacctagatgtccctatcgcgtgtacactcg

SEQ ID NO: 62 - DNA, 27 bp, Artificial Sequence
  Artificial DNA Primer
taggtcgactctagcgagctcgagatc

SEQ ID NO: 63 - DNA, 40 bp, Artificial Sequence
  Artificial DNA Primer
catggccagttgtgtatatagaggattgaggaaggaagag

SEQ ID NO: 64 - DNA, 11101 bp, Artificial Sequence
  Artificial DNA Sequence
ttgaagttcctattccgagttcctattctctagaaagtataggaacttcagtacccgggtataagctagcttccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagtttccaactcaatttacctctatccacacttctcttccttcctcaatcctctatatacacaactggggatccaccatgttctcggcaggccacaagattaagggtacagtcgtcctcatgcctaaaaacgagttggaagtgaaccccgatggctccgcagtcgataacctcaacgcattcctcggacgttcggtgtcgctccagctcatctccgcgaccaaagccgacgcccacggtaagggaaaggtgggcaaggacacgttcttggaaggtatcaacacttcgctccctaccttgggagcaggagagtccgcattcaacattcacttcgagtgggacggttcgatgggcattcccggagcgttctatatcaagaactatatgcaggtggagttcttcttgaagtccttgaccttggaggcaatctcgaaccagggtaccatccgtttcgtgtgtaactcgtgggtctacaacaccaagctctacaaatccgtgcggatcttcttcgcgaaccacacttacgtcccttcggagacacctgcccctttggtgtcgtaccgcgaggaggaattgaagtccctccgtggtaacggtactggagaaaggaaggagtatgataggatctacgactacgacgtctataacgatttgggtaaccccgacaaatcggaaaagttggcacgtcctgtgttgggaggctcctccaccttcccctaccctcgacgcggccgcacgggacgcggtcccactgtcaccgatccgaacacagagaagcagggcgaagtcttctacgtgcccagggacgaaaacctcggccacttgaagtcgaaggatgcattggagattggaaccaagtccctctcccagatcgtccagcctgcattcgaatcggcgttcgatttgaaatcgacgcccatcgagttccactcgttccaggacgtccatgacttgtatgaaggtggtatcaaattgcctcgggacgtcatctccaccattatccccctccccgtgatcaaggaattgtaccgcaccgacggccagcatattctcaaattcccccagccgcacgtcgtccaggtctcgcagtccgcatggatgacagatgaggaattcgcgagggaaatgattgcaggtgtcaacccgtgtgtcatccgaggcttggaggagttccctcctaagtccaacctcgatcctgccatctatggagaccagtcctccaagattacagccgattccctcgatctcgacggttatactatggatgaagcactcggttccaggcgattgttcatgctcgattatcatgatatcttcatgccctatgtgcgccagatcaaccagttgaactcggcaaaaacatatgcaacgaggacgatcctcttcctccgagaagacggcacactcaagcctgtggcaatcgagctctcgctcccccattccgcaggcgatctctccgcagccgtgtcgcaggtggtgttgcctgcaaaagaaggagtggagtcgaccatctggctcttggccaaagcatatgtgattgtgaacgattcctgttatcaccagctcatgtcgcattggctcaacactcacgcggcaatggaacccttcgtgatcgccacgcaccggcacctctcggtgctccacccgatctacaagctcctcactccccactaccgtaacaacatgaacattaacgccttggcacggcagtcgttgatcaacgcgaacggcatcattgagacaacgttcctcccctccaagtactccgtcgaaatgtcgtccgcagtctacaaaaactgggtcttcaccgaccaggcgttgcctgccgacttgatcaaacgaggcgtcgcaatcaaagatccctccactcctcatggcgtccgcctcttgatcgaggactacccctacgcagcggacggattggaaatctgggcagccatcaagacctgggtgcaggaatacgtccctttgtactatgcgagggacgatgatgtcaaaaacgactcggaactccagcattggtggaaggaggcagtggaaaagggccatggagatctcaaggataaaccctggtggcctaagctccagaccttggaggacctcgtcgaagtgtgtttgatcattatctggatcgcatccgcgttgcatgcagccgtgaacttcggacagtatccctatggaggcctcatcatgaaccgtcccaccgcatccaggaggctcctccccgaaaaaggaacacccgaatacgaagaaatgatcaacaaccacgaaaaggcatacctccggaccatcacttccaaactcccgaccttgatctcgctctccgtgatcgagattttgtcgacacatgcgtcggacgaggtctatttgggtcagcgggataacccgcactggacatccgattccaaggccctccaggcgttccagaagttcggcaacaagctcaaggagatcgaggagaaactcgtgaggcggaacaacgacccttccctccagggaaaccggttgggacctgtccagctcccgtatacgttgctctacccctcctcggaagaaggcctcactttcaggggtatccccaactcgatttccatctgactcgagatctagagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggccgggtggtcattggccgcctgtgatgccatctgccactaaatccgatcattgatccaccgcccacgaggcgcgtctttgctttttgcgcggcgtccaggttcaactctctcttaattaaatagcgacaagccgaacggcaccggcaggtacaatggttcgctgtacttgcttgcgcaagcgggtctttggggattgagcgcatttggtgttgcaaaggatttgatgtaaatgtagtcgacatcttagcacagaggggagagttgataaaatgtggtctgtttgaatgatagtcgggttcgtgacctatattcgtgatagtggagataggtctgcgcctatcttatcgggccggagcaaaaattccaccgcagcggggtgagttttcgttatacagccatcccacttccagcttcaaattgtcagtttaatccagcccaattcaatcattggagaaccggttttatgtcttcgaagtcccacctcccctacgcaattcgcgcaaccaaccatcccaaccctttaacatctaaactcttctccatcgccgaggagaagaaaaccaacgtcaccgtctccgcagacgttactacttccgccgagctcctcgatcttgctgaccgcctaggcccctatatcgcagttctgaaaacccacatcgacatcctcaccgatctcaccccgtcgaccctttcctcgctccaatccctcgcgacaaagcacaacttcctcatctttgaggaccgcaagttcatcgacatcggcaacaccgtgcaaaagcagtaccacggtggcgctctccgcatctccgaatgggcacacatcatcaactgcgccatcctgccgggcgaagggatcgtcgaggccctcgcacagacaaccaagtctcctgactttaaagacgcgaatcaacgaggtctcctgattcttgccgagatgacgagtaagggatctcttgcgacaggggagtacacggcacgctcggttgagtacgcgcggaagtataaggggtttgtgatgggattcgtgagtacaagggcgttgagtgaggtgctgcccgaacagaaagaggagagcgaggattttgtcgtctttacgactggggtgaatctgtcggataagggggataagctggggcagcagtatcagacacctgggtcggcggttgggcgaggtgcggactttatcattgcgggtaggggcatctataaggcggacgatccagtcgaggcggttcagaggtaccgggaggaaggctggaaagcttacgagaaaagagttggactttgagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggccgggtggtcattggccgcctgtgatgccatctgccactaaatccgatcattgatccaccgcccacgaggcgcgtctttgctttttgcgcggcgtccaggttcaactctctcctctaggttgaagttcctattccgagttcctattcttcaaatagtataggaacttcaactagctagtgcatgcgtacgattttgacatttgctccattgtcgaggatggatggaacgagcggcgtgcgccacgaaagtgaggctattgcctatcagctctttgctacattccggaaacaaacatccctttttgtgaattatctacgcaacttagatggcgtgaacgcatcttcaaagtctttcggcaggtccggcacgacttttgcatccagagaagcgcctacatgtgtattcgaccacctcctagcgcgcttggatatgaggaaatattactgagagtcgaaaacaagctccaccgcaccagctcttcttggagttttatattaaagaatattcccagctcgttgtattattctttttctaccgtgctaatgtatcaaggactttggtacctattaacgttattattcgtgtgctattcccaaacataaccctgtatatgtttcgaacgccgttatgacccatgtcttacatactcattaagtcattcccttggataatctcgactcagatgcggcggttgatgtaggaggagaggtaatcgaggacctcctgggagatgatgccgttccaggcggggtagcggatggagccctcggcggagcccttgagctgctcgatatgctgccactcctcgatggggttggtctcatccttgagggcgatcatctccttggagatgggatcgtaggcgtagtagcgggagactagtgcgaagtaatgatcggggatggcggtgatctgatgggtgtaggtggtgcgggcgacggcggaggcgcgcttatcggaccagttgccgacgacgttggtgagctcggtgaggcccttcatggagaggaaggaggtcatgagatggcggccgatatgggacttggggccgttcttgatggcgaagatggagtagggggcgttcttcttgagggccttgttgtaggagcggacgaggttatccttgaggagctggtactcctgcttgttggaggaggagttgccggtgcggttgacgcgcttgaggacgggctcggagttgcggaggaactcatcgaggtagacgaggggatcgatgcggccgcgggcggagaagaagtagatatggcgggagacggaggtcttggtctcggtgacgaggcactggatgatgacgccgaggtacttgttctggacgagcttgaaggacttgggatcgacgttcttgatatcggagaagcggccgcagttgatgaaggtggcgaggaagaggaactggtagagggtcttggtcttggtgaagcgggaggtgtactcgaaggagttgaggatcttctcggtgatctcccagatggactcgccctcggagaggagggccttgagcatcttcttggaatgggagttgcccttatcggcctcctcggaggactcgaactggagctggagggaggagacgatatcggtgatatcggactgatgcttctggccgtagtaggggatgatggtgaactcccaggcggggatgagcttcttgagggaggcctccaggatggtggccttctgggtcttgtacttgaactggagggacttgttgacgatatcgaaggagagggagttggagatgatggtgttgtaggacatgaaggtggcgcgcttgatggcggtgccgttatgggtgatcatccagcagaggtaggtgagctcggcggcgcagagggcgatcttctcgccggaggggcgctcgaagcgctcgacgaactggcggacgaggaccttggggggggtcttgcagaggatatcgaactggggcatggtgctcagatactacggctgatcgcgtagaggtactgagcaaaacagatgtcagtaaggagaagagttgaatgaatggaagaagagtaggaaaggaggtatgggggaaagatatacgtactgatgcggacgaagagagaaagaaggaaaaaagttgtgggaggggaaggagggggaatccttatatggaggggcaagcgagaaggcgaattagtgggcgggcttaagccctcgaccgccgcccttatcattggacatggaggggtaatgcccccaccacgcatgtgcgggaccgacgcagaatctgcacggcggagtctcttccagactgttgacttttgggcgatgactcttgttgctgcggccttttgggtacaccaacctcgttgatcttgtttccttggttctctttcgctcggagacccgaccatgaccccaccatcagtcactatcctgcctcgtcgataaaaattttttcttccctctgattgttacatagtatgtttccacctttccggtggatttcggacagtcaaactgggcatcaacgcagtggtgggctgcttcgtttgctgcgtgttgtacttgtttgcatttgaaccccgcggtcgttcgagtccttaattggtccgctcccggtcaacacccaagcagctgtggcccggccgagtggcgcctgtctggtccacagtaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttttcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacc

SEQ ID NO: 65 - DNA, 50 bp, Artificial Sequence
  Artificial DNA Primer
accgggaggaaggctggaaagcttacgagaaaagagttggactttgaggg

SEQ ID NO: 66 - DNA, 40 bp, Artificial Sequence
  Artificial DNA Primer
tgagcgaggaagcggaagagcgcccaatacgcaaaccgcc

SEQ ID NO: 67 - DNA, 40 bp, Artificial Sequence
  Artificial DNA Primer
tgcgtattgggcgctcttccgcttcctcgctcactgactc

SEQ ID NO: 68 - DNA, 68 bp, Artificial Sequence
  Artificial DNA Primer
tatactttctagagaataggaactcggaataggaacttcaaggaacaacactcaaccctatctcggtc

SEQ ID NO: 69 - DNA, 67 bp, Artificial Sequence
  Artificial DNA Primer
tccgagttcctattctctagaaagtataggaacttcgcatttatcagggttattgtctcatgagcgg

SEQ ID NO: 70 - DNA, 41 bp, Artificial Sequence
  Artificial DNA Primer
tctagatctcgagtcagatgtccctatcgcgtgtacactcg

SEQ ID NO: 71 - DNA, 45 bp, Artificial Sequence
  Artificial DNA Primer
acacgcgatagggacatctgactcgagatctagagggtgactgac

SEQ ID NO: 72 - DNA, 40 bp, Artificial Sequence
  Artificial DNA Primer
aactcttttctcgtaagctttccagccttcctcccggtac

(19) United States
(12) Patent Application Publication
(10) Pub. No.: US 2017/0267980 A1
(43) Pub. Date: Sep. 21, 2017

(54) Xyloglucan Endotransglycosylase variants and Polynucleotides Encoding Same

(71) Applicant: Novozymes A/S, Bagsvaerd (DK)

(72) Inventor: Daniel Held, Davis, CA (US)

(73) Assignee: Novozymes A/S, Bagsvaerd (DK)

(21) Appl. No.: 15/505,168

(22) PCT Filed: Aug. 20, 2015
(86) PCT No.: PCT/US2015/046083

§ 371 Date: Feb. 20, 2017

Related U.S. Application Data
(60) Provisional application No. 62/039,744, filed on Aug. 20, 2014.

(51) Int. Cl. C12N 9/10

(52) U.S. Cl. CPC C12N 9/1051; C12Y 204/01207

(57) ABSTRACT

The present invention relates to xyloglucan endotransglycosylase variants. The present invention also relates to polynucleotides encoding the variants; nucleic acid constructs, vectors, and host cells comprising the polynucleotides; and methods of using the variants.

### REFERENCE TO A SEQUENCE LISTING

This application contains a Sequence Listing in computer readable form, which is incorporated herein by reference.

### BACKGROUND OF THE INVENTION

Field of the Invention

The present invention relates to xyloglucan endotransglycosylase variants, polynucleotides encoding the variants, methods of producing the variants, and methods of using the variants.

Description of the Related Art

Xyloglucan endotransglycosylase (XET) is an enzyme that catalyzes endotransglycosylation of xyloglucan, a structural polysaccharide of plant cell walls. The enzyme is present in most plants, and in particular, land plants. XET has been extracted from dicotyledons and monocotyledons.

Heterologous expression of xyloglucan endotransglycosylases at commercially-relevant levels in industrial microorganisms has not yet been achieved. There is a need in the art to improve expression of xyloglucan endotransglycosylases in industrially important microorganisms.

The present invention provides xyloglucan endotransglycosylase variants with increased expression yield compared to its parent.

### SUMMARY OF THE INVENTION

The present invention relates to isolated xyloglucan endotransglycosylase variants, comprising a substitution at one or more (e.g., several) positions corresponding to positions 10, 30, 40, 51, 53, 60, 99, 102, 117, 130, 136, 157, 162, 175, 183, 254, and 280 of the full-length polypeptide of SEQ ID NO: 2, wherein the variants have xyloglucan endotransglycosylase activity.

The present invention also relates to isolated polynucleotides encoding the variants; nucleic acid constructs, vectors, and host cells comprising the polynucleotides; and methods of producing the variants.

The present invention also relates to methods for obtaining the variants and methods of increasing the expression yield of a xyloglucan endotransglycosylase.

The present invention further relates to compositions comprising the variants.

### BRIEF DESCRIPTION OF THE FIGURES

FIG. 1 shows a restriction map of pMMar27.

FIG. 2 shows a restriction map of pEvFz1.

FIG. 3 shows a restriction map of pDLHD0006.

FIG. 4 shows a restriction map of pDLHD0044.

FIG. 5 shows a restriction map of pDau571.

FIG. 6 shows a restriction map of pDLHD0075.

FIG. 7 shows a restriction map of pDLHD0095.

### DEFINITIONS

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which begins with a start codon, such as ATG, GTG or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Fragment: The term “fragment” means a polypeptide having one or more (e.g., several) amino acids absent from the amino and/or carboxyl terminus of the mature polypeptide thereof, wherein the fragment has xyloglucan endotransglycosylase activity. In one aspect, a fragment contains at least 85%, at least 90%, or at least 95% of the amino acid residues of the mature polypeptide.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” means a characteristic associated with a variant that is improved compared to the parent. In the present invention, the improved property is increased expression yield of a variant relative to the parent.

Increased expression yield: The term “increased expression yield” means a higher amount (g) of secreted active enzyme per liter of culture medium from cultivation of a host cell expressing the variant gene relative to the amount (g) of secreted active enzyme per liter produced under the same cultivation conditions by the same host cell expressing the parent gene. In one aspect, the variant has an increased expression yield compared to the parent enzyme of at least 1.05, at least 1.10, at least 1.20, at least 1.30, at least 1.40, at least 1.50, at least 1.60, at least 1.70, at least 1.80, at least 1.90, at least 2, at least 2.25, at least 2.50, at least 2.75, at least 3.00, at least 3.25, at least 3.50, at least 3.75, at least 4, at least 4.25, at least 4.50, at least 4.75, at least 5, at least 6, at least 7, at least 8, at least 9, or at least 10 fold.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 21 to 292 of SEQ ID NO: 2 based on the SignalP 3.0 program (Bendtsen et al., 2004, J. Mol. Biol. 340: 783-795) that predicts amino acids 1 to 20 of SEQ ID NO: 2 are a signal peptide. In another aspect, the mature polypeptide is amino acids 28 to 287 of SEQ ID NO: 4 based on the SignalP 3.0 program that predicts amino acids 1 to 27 of SEQ ID NO: 4 are a signal peptide. In another aspect, the mature polypeptide is amino acids 23 to 294 of SEQ ID NO: 6 based on the SignalP 3.0 program that predicts amino acids 1 to 22 of SEQ ID NO: 6 are a signal peptide. In another aspect, the mature polypeptide is amino acids 25 to 297 of SEQ ID NO: 8 based on the SignalP 3.0 program that predicts amino acids 1 to 24 of SEQ ID NO: 8 are a signal peptide. In another aspect, the mature polypeptide is amino acids 23 to 294 of SEQ ID NO: 10 based on the SignalP 3.0 program that predicts amino acids 1 to 22 of SEQ ID NO: 10 are a signal peptide. In another aspect, the mature polypeptide is amino acids 27 to 285 of SEQ ID NO: 12 based on the SignalP 3.0 program that predicts amino acids 1 to 26 of SEQ ID NO: 12 are a signal peptide. In another aspect, the mature polypeptide is amino acids 23 to 323 of SEQ ID NO: 14 based on the SignalP 3.0 program that predicts amino acids 1 to 22 of SEQ ID NO: 14 are a signal peptide. In another aspect, the mature polypeptide is amino acids 29 to 299 of SEQ ID NO: 16 based on the SignalP 3.0 program that predicts amino acids 1 to 28 of SEQ ID NO: 16 are a signal peptide. In another aspect, the mature polypeptide is amino acids 25 to 275 of SEQ ID NO: 18 based on the SignalP 3.0 program that predicts amino acids 1 to 24 of SEQ ID NO: 18 are a signal peptide. In another aspect, the mature polypeptide is amino acids 19 to 330 of SEQ ID NO: 20 based on the SignalP 3.0 program that predicts amino acids 1 to 18 of SEQ ID NO: 20 are a signal peptide. In another aspect, the mature polypeptide is amino acids 25 to 297 of SEQ ID NO: 22 based on the SignalP 3.0 program that predicts amino acids 1 to 24 of SEQ ID NO: 22 are a signal peptide. In another aspect, the mature polypeptide is amino acids 25 to 288 of SEQ ID NO: 24 based on the SignalP 3.0 program that predicts amino acids 1 to 24 of SEQ ID NO: 24 are a signal peptide. In another aspect, the mature polypeptide is amino acids 25 to 311 of SEQ ID NO: 26 based on the SignalP 3.0 program that predicts amino acids 1 to 24 of SEQ ID NO: 26 are a signal peptide. In another aspect, the mature polypeptide is amino acids 29 to 291 of SEQ ID NO: 28 based on the SignalP 3.0 program that predicts amino acids 1 to 28 of SEQ ID NO: 28 are a signal peptide. In another aspect, the mature polypeptide is amino acids 26 to 280 of SEQ ID NO: 30 based on the SignalP 3.0 program that predicts amino acids 1 to 25 of SEQ ID NO: 30 are a signal peptide. In another aspect, the mature polypeptide is amino acids 22 to 290 of SEQ ID NO: 32 based on the SignalP 3.0 program that predicts amino acids 1 to 21 of SEQ ID NO: 32 are a signal peptide. In another aspect, the mature polypeptide is amino acids 31 to 302 of SEQ ID NO: 34 based on the SignalP 3.0 program that predicts amino acids 1 to 30 of SEQ ID NO: 34 are a signal peptide. In another aspect, the mature polypeptide is amino acids 28 to 291 of SEQ ID NO: 36 based on the SignalP 3.0 program that predicts amino acids 1 to 27 of SEQ ID NO: 36 are a signal peptide. In another aspect, the mature polypeptide is amino acids 30 to 299 of SEQ ID NO: 38 based on the SignalP 3.0 program that predicts amino acids 1 to 29 of SEQ ID NO: 38 are a signal peptide. In another aspect, the mature polypeptide is amino acids 27 to 290 of SEQ ID NO: 40 based on the SignalP 3.0 program that predicts amino acids 1 to 26 of SEQ ID NO: 40 are a signal peptide. In another aspect, the mature polypeptide is amino acids 29 to 304 of SEQ ID NO: 42 based on the SignalP 3.0 program that predicts amino acids 1 to 28 of SEQ ID NO: 42 are a signal peptide. In another aspect, the mature polypeptide is amino acids 22 to 292 of SEQ ID NO: 44 based on the SignalP 3.0 program that predicts amino acids 1 to 21 of SEQ ID NO: 44 are a signal peptide. In another aspect, the mature polypeptide is amino acids 31 to 283 of SEQ ID NO: 46 based on the SignalP 3.0 program that predicts amino acids 1 to 30 of SEQ ID NO: 46 are a signal peptide. In another aspect, the mature polypeptide is amino acids 23 to 283 of SEQ ID NO: 48 based on the SignalP 3.0 program that predicts amino acids 1 to 22 of SEQ ID NO: 48 are a signal peptide. In another aspect, the mature polypeptide is amino acids 29 to 290 of SEQ ID NO: 50 based on the SignalP 3.0 program that predicts amino acids 1 to 28 of SEQ ID NO: 50 are a signal peptide.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having xyloglucan endotransglycosylase activity. In one aspect, the mature polypeptide coding sequence is nucleotides 61 to 876 of SEQ ID NO: 1 based on the SignalP 3.0 program that predicts nucleotides 1 to 60 of SEQ ID NO: 1 encode a signal peptide. In another aspect, the mature polypeptide coding sequence is nucleotides 82 to 861 of SEQ ID NO: 3 based on the SignalP 3.0 program that predicts nucleotides 1 to 81 of SEQ ID NO: 3 encode a signal peptide. In another aspect, the mature polypeptide coding sequence is nucleotides 67 to 882 of SEQ ID NO: 5 based on the SignalP 3.0 program that predicts nucleotides 1 to 66 of SEQ ID NO: 5 encode a signal peptide. In another aspect, the mature polypeptide coding sequence is nucleotides 73 to 891 of SEQ ID NO: 7 based on the SignalP 3.0 program that predicts nucleotides 1 to 72 of SEQ ID NO: 7 encode a signal peptide. In another aspect, the mature polypeptide coding sequence is nucleotides 67 to 882 of SEQ ID NO: 9 based on the SignalP 3.0 program that predicts nucleotides 1 to 66 of SEQ ID NO: 9 encode a signal peptide. In another aspect, the mature polypeptide coding sequence is nucleotides 79 to 855 of SEQ ID NO: 11 based on the SignalP 3.0 program that predicts nucleotides 1 to 78 of SEQ ID NO: 11 encode a signal peptide. In another aspect, the mature polypeptide coding sequence is nucleotides 67 to 969 of SEQ ID NO: 13 based on the SignalP 3.0 program that predicts nucleotides 1 to 66 of SEQ ID NO: 13 encode a signal peptide. In another aspect, the mature polypeptide coding sequence is nucleotides 85 to 897 of SEQ ID NO: 15 based on the SignalP 3.0 program that predicts nucleotides 1 to 84 of SEQ ID NO: 15 encode a signal peptide. In another aspect, the mature polypeptide coding sequence is nucleotides 73 to 825 of SEQ ID NO: 17 based on the SignalP 3.0 program that predicts nucleotides 1 to 72 of SEQ ID NO: 17 encode a signal peptide. In another aspect, the mature polypeptide coding sequence is nucleotides 55 to 990 of SEQ ID NO: 19 based on the SignalP 3.0 program that predicts nucleotides 1 to 54 of SEQ ID NO: 19 encode a signal peptide. In another aspect, the mature polypeptide coding sequence is nucleotides 73 to 891 of SEQ ID NO: 21 based on the SignalP 3.0 program that predicts nucleotides 1 to 72 of SEQ ID NO: 21 encode a signal peptide. In another aspect, the mature polypeptide coding sequence is nucleotides 73 to 864 of SEQ ID NO: 23 based on the SignalP 3.0 program that predicts nucleotides 1 to 72 of SEQ ID NO: 23 encode a signal peptide. In another aspect, the mature polypeptide coding sequence is nucleotides 73 to 933 of SEQ ID NO: 25 based on the SignalP 3.0 program that predicts nucleotides 1 to 72 of SEQ ID NO: 25 encode a signal peptide. In another aspect, the mature polypeptide coding sequence is nucleotides 85 to 873 of SEQ ID NO: 27 based on the SignalP 3.0 program that predicts nucleotides 1 to 84 of SEQ ID NO: 27 encode a signal peptide. In another aspect, the mature polypeptide coding sequence is nucleotides 76 to 840 of SEQ ID NO: 29 based on the SignalP 3.0 program that predicts nucleotides 1 to 75 of SEQ ID NO: 29 encode a signal peptide. In another aspect, the mature polypeptide coding sequence is nucleotides 64 to 870 of SEQ ID NO: 31 based on the SignalP 3.0 program that predicts nucleotides 1 to 63 of SEQ ID NO: 31 encode a signal peptide. In another aspect, the mature polypeptide coding sequence is nucleotides 91 to 906 of SEQ ID NO: 33 based on the SignalP 3.0 program that predicts nucleotides 1 to 90 of SEQ ID NO: 33 encode a signal peptide. In another aspect, the mature polypeptide coding sequence is nucleotides 82 to 873 of SEQ ID NO: 35 based on the SignalP 3.0 program that predicts nucleotides 1 to 81 of SEQ ID NO: 35 encode a signal peptide. In another aspect, the mature polypeptide coding sequence is nucleotides 88 to 897 of SEQ ID NO: 37 based on the SignalP 3.0 program that predicts nucleotides 1 to 87 of SEQ ID NO: 37 encode a signal peptide. In another aspect, the mature polypeptide coding sequence is nucleotides 79 to 870 of SEQ ID NO: 39 based on the SignalP 3.0 program that predicts nucleotides 1 to 78 of SEQ ID NO: 39 encode a signal peptide. In another aspect, the mature polypeptide coding sequence is nucleotides 85 to 912 of SEQ ID NO: 41 based on the SignalP 3.0 program that predicts nucleotides 1 to 84 of SEQ ID NO: 41 encode a signal peptide. In another aspect, the mature polypeptide coding sequence is nucleotides 64 to 876 of SEQ ID NO: 43 based on the SignalP 3.0 program that predicts nucleotides 1 to 63 of SEQ ID NO: 43 encode a signal peptide. In another aspect, the mature polypeptide coding sequence is nucleotides 91 to 849 of SEQ ID NO: 45 based on the SignalP 3.0 program that predicts nucleotides 1 to 90 of SEQ ID NO: 45 encode a signal peptide. In another aspect, the mature polypeptide coding sequence is nucleotides 67 to 849 of SEQ ID NO: 47 based on the SignalP 3.0 program that predicts nucleotides 1 to 66 of SEQ ID NO: 47 encode a signal peptide. In another aspect, the mature polypeptide coding sequence is nucleotides 85 to 870 of SEQ ID NO: 49 based on the SignalP 3.0 program that predicts nucleotides 1 to 84 of SEQ ID NO: 49 encode a signal peptide. In each of the aspects above, the term “mature polypeptide coding sequence” shall be understood to include the cDNA sequence of the genomic DNA sequence or the genomic DNA sequence of the cDNA sequence.

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent xyloglucan endotransglycosylase: The term “parent” or “parent xyloglucan endotransglycosylase” means a xyloglucan endotransglycosylase to which an alteration, i.e., a substitution, insertion, and/or deletion, at one or more (e.g., several) positions, is made to produce the xyloglucan endotransglycosylase variants of the present invention. The parent may be a naturally occurring (wild-type) polypeptide or a variant or fragment thereof.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are a gap open penalty of 10, a gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are a gap open penalty of 10, a gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Subsequence: The term “subsequence” means a polynucleotide having one or more (e.g., several) nucleotides absent from the 5′ and/or 3′ end of a mature polypeptide coding sequence, wherein the subsequence encodes a fragment having xyloglucan endotransglycosylase activity. In one aspect, a subsequence contains at least 85%, at least 90%, or at least 95% of the nucleotides of the mature polypeptide coding sequence.

Variant: The term “variant” means a polypeptide having xyloglucan endotransglycosylase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. The variants of the present invention have at least 20%, e.g., at least 40%, at least 50%, at least 60%, at least 70%, at least 80%, at least 90%, at least 95%, or at least 100% of the xyloglucan endotransglycosylase activity of the parent.

Wild-type xyloglucan endotransglycosylase: The term “wild-type” xyloglucan endotransglycosylase means a xyloglucan endotransglycosylase expressed by a naturally occurring organism, such as a plant, bacterium, yeast, or filamentous fungus found in nature.

Xyloglucan endotransglycosylase: The term “xyloglucan endotransglycosylase” means a xyloglucan:xyloglucan xyloglucanotransferase (EC 2.4.1.207) that catalyzes cleavage of a β-(1→4) bond in the backbone of a xyloglucan and transfers the xyloglucanyl segment on to 0-4 of the non-reducing terminal glucose residue of an acceptor, which can be a xyloglucan or an oligosaccharide of xyloglucan. Xyloglucan endotransglycosylases are also known as xyloglucan endotransglycosylase/hydrolases or endo-xyloglucan transferases. Some xylan endotransglycosylases can possess different activities including xyloglucan and mannan endotransglycosylase activities. For example, xylan endotransglycosylase from ripe papaya fruit can use heteroxylans, such as wheat arabinoxylan, birchwood glucuronoxylan, and others as donor molecules. These xylans could potentially play a similar role as xyloglucan while being much cheaper in cost since they can be extracted, for example, from pulp mill spent liquors and/or future biomass biorefineries.

Xyloglucan endotransglycosylase activity can be assayed by those skilled in the art using any of the following methods. Reduction of average molecular weight of a xyloglucan polymer by incubation of the xyloglucan polymer with a molar excess of a xyloglucan oligomer in the presence of xyloglucan endotransglycosylase can be determined via liquid chromatography (Sulova et al., 2003, Plant Physiol. Biochem. 41: 431-437) or via ethanol precipitation (Yaanaka et al., 2000, Food Hydrocolloids 14: 125-128) followed by gravimetric or cellulose-binding analysis (Fry et al., 1992, Biochem. J. 282: 821-828), or can be assessed colorimetrically by association with iodine under alkaline conditions (Sulova et al., 1995, Analytical Biochemistry 229: 80-85).

### Conventions for Designation of Variants

For purposes of the present invention, the full-length xyloglucan endotransglycosylase disclosed in SEQ ID NO: 2 is used to determine the corresponding amino acid residue in another xyloglucan endotransglycosylase. The amino acid sequence of another xyloglucan endotransglycosylase is aligned with the full-length polypeptide disclosed in SEQ ID NO: 2, and based on the alignment, the amino acid position number corresponding to any amino acid residue in the full-length polypeptide of SEQ ID NO: 2 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are a gap open penalty of 10, a gap extension penalty of 0.5, and the EBLOSUM62 (EMBOSS version of BLOSUM62) substitution matrix. Numbering of the amino acid positions is based on the full-length polypeptide (e.g., including the signal peptide) of SEQ ID NO: 2 wherein position 1 is the first amino acid of the signal peptide (i.e., Met) and position 21 (i.e., Ala) is the first position of the mature polypeptide of SEQ ID NO: 2.

Identification of the corresponding amino acid residue in another xyloglucan endotransglycosylase can be determined by alignment of multiple polypeptide sequences using several computer programs including, but not limited to, MUSCLE (multiple sequence comparison by log-expectation; version 3.5 or later; Edgar, 2004, Nucleic Acids Research 32: 1792-2797), MAFTT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When another xyloglucan endotransglycosylase has diverged from the full-length polypeptide of SEQ ID NO: 2 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411 Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates to isolated xyloglucan endotransglycosylase variants, comprising a substitution at one or more (e.g., several) positions corresponding to positions 10, 30, 40, 51, 53, 60, 99, 102, 117, 130, 136, 157, 162, 175, 183, 254, and 280 of the full-length polypeptide of SEQ ID NO: 2, wherein the variants have xyloglucan endotransglycosylase activity.

### Variants

In an embodiment, the variants have a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, to the amino acid sequence of the parent xyloglucan endotransglycosylase.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 2.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 4.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 6.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 8.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 10.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 12.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 14.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 16.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 18.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 20.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 22.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 24.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 26.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 28.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 30.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 32.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 34.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 36.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 38.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 40.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 42.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 44.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 46.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 48.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 50.

In one aspect, the number of substitutions in the variants of the present invention is 1-17, e.g., 1, 2, 3, 4, 5, 6, 7, 8, 9, 10, 11, 12, 13, 14, 15, 16, or 17 substitutions.

In another aspect, a variant comprises a substitution at one or more (e.g., several) positions corresponding to positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at two positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at three positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at four positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at five positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at six positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at seven positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at eight positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at nine positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at ten positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at eleven positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at twelve positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at thirteen positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at fourteen positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at fifteen positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at sixteen positions corresponding to any of positions 10, 30, 40, 51, 53, 60, 99, 102, 117, 130, 136, 157, 162, 175, 183, 254, and 280 of the full-length polypeptide of SEQ ID NO: 2. In another aspect, a variant comprises a substitution at each position corresponding to positions 10, 30, 40, 51, 53, 60, 99, 102, 117, 130, 136, 157, 162, 175, 183, 254, and 280 of the full-length polypeptide of SEQ ID NO: 2.

In another aspect, the variant comprises or consists of a substitution at a position corresponding to position 10. In another aspect, the amino acid at a position corresponding to position 10 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala. In another aspect, the variant comprises or consists of the substitution I10A of the full-length polypeptide of SEQ ID NO: 2.

In another aspect, the variant comprises or consists of a substitution at a position corresponding to position 30. In another aspect, the amino acid at a position corresponding to position 30 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu. In another aspect, the variant comprises or consists of the substitution P30 E of the full-length polypeptide of SEQ ID NO: 2.

In another aspect, the variant comprises or consists of a substitution at a position corresponding to position 40. In another aspect, the amino acid at a position corresponding to position 40 is substituted with Ala, Arg, Asn, Asp, Cys, Gin, Glu, Gly, His, Ile, Leu, Lys, Met, Phe, Pro, Ser, Thr, Trp, Tyr, or Val, preferably with Gly. In another aspect, the variant comprises or consists of the substitution A40G of the full-length polypeptide of SEQ ID NO: 2.

In another aspect, the variant comprises or consists of a substitution at a position corresponding to position 51. In another aspect, the amino acid at a position corresponding to position 51 is substituted with Ala, Arg, Asn, Asp, Cys, Gln, Glu, Gly, His, Ile, Leu, Lys, Met, Phe, Pro, Ser, Thr, Trp, Tyr, or Val, preferably with Thr. In another aspect, the variant comprises or consists of the substitution S51T of the full-length polypeptide of SEQ ID NO: 2.

In another aspect, the variant comprises or consists of a substitution at a position corresponding to position 53. In another aspect, the amino acid at a position corresponding to position 53 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala or Val. In another aspect, the variant comprises or consists of the substitution I53A,V of the full-length polypeptide of SEQ ID NO: 2.

In another aspect, the variant comprises or consists of a substitution at a position corresponding to position 60. In another aspect, the amino acid at a position corresponding to position 60 is substituted with Ala, Arg, Asn, Asp, Cys, Gin, Glu, Gly, His, Ile, Leu, Lys, Met, Phe, Pro, Ser, Thr, Trp, Tyr, or Val, preferably with Ser. In another aspect, the variant comprises or consists of the substitution Y60S of the full-length polypeptide of SEQ ID NO: 2.

In another aspect, the variant comprises or consists of a substitution at a position corresponding to position 99. In another aspect, the amino acid at a position corresponding to position 99 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu or Asn. In another aspect, the variant comprises or consists of the substitution T99E,N of the full-length polypeptide of SEQ ID NO: 2.

In another aspect, the variant comprises or consists of a substitution at a position corresponding to position 102. In another aspect, the amino acid at a position corresponding to position 102 is substituted with Ala, Arg, Asn, Asp, Cys, Gln, Glu, Gly, His, Ile, Leu, Lys, Met, Phe, Pro, Ser, Thr, Trp, Tyr, or Val, preferably with Gly. In another aspect, the variant comprises or consists of the substitution E102G of the full-length polypeptide of SEQ ID NO: 2.

In another aspect, the variant comprises or consists of a substitution at a position corresponding to position 117. In another aspect, the amino acid at a position corresponding to position 117 is substituted with Ala, Arg, Asn, Asp, Cys, Gin, Glu, Gly, His, Ile, Leu, Lys, Met, Phe, Pro, Ser, Thr, Trp, Tyr, or Val, preferably with Glu. In another aspect, the variant comprises or consists of the substitution Q117E of the full-length polypeptide of SEQ ID NO: 2.

In another aspect, the variant comprises or consists of a substitution at a position corresponding to position 130. In another aspect, the amino acid at a position corresponding to position 130 is substituted with Ala, Arg, Asn, Asp, Cys, Gln, Glu, Gly, His, Ile, Leu, Lys, Met, Phe, Pro, Ser, Thr, Trp, Tyr, or Val, preferably with Arg. In another aspect, the variant comprises or consists of the substitution K130R of the full-length polypeptide of SEQ ID NO: 2.

In another aspect, the variant comprises or consists of a substitution at a position corresponding to position 136. In another aspect, the amino acid at a position corresponding to position 136 is substituted with Ala, Arg, Asn, Asp, Cys, Gln, Glu, Gly, His, Ile, Leu, Lys, Met, Phe, Pro, Ser, Thr, Trp, Tyr, or Val, preferably with Trp. In another aspect, the variant comprises or consists of the substitution R136W of the full-length polypeptide of SEQ ID NO: 2.

In another aspect, the variant comprises or consists of a substitution at a position corresponding to position 157. In another aspect, the amino acid at a position corresponding to position 157 is substituted with Ala, Arg, Asn, Asp, Cys, Gln, Glu, Gly, His, Ile, Leu, Lys, Met, Phe, Pro, Ser, Thr, Trp, Tyr, or Val, preferably with His. In another aspect, the variant comprises or consists of the substitution Y157H of the full-length polypeptide of SEQ ID NO: 2.

In another aspect, the variant comprises or consists of a substitution at a position corresponding to position 162. In another aspect, the amino acid at a position corresponding to position 162 is substituted with Ala, Arg, Asn, Asp, Cys, Gin, Glu, Gly, His, Ile, Leu, Lys, Met, Phe, Pro, Ser, Thr, Trp, Tyr, or Val, preferably with Cys. In another aspect, the variant comprises or consists of the substitution Y162C of the full-length polypeptide of SEQ ID NO: 2.

In another aspect, the variant comprises or consists of a substitution at a position corresponding to position 175. In another aspect, the amino acid at a position corresponding to position 175 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser, Gly, or Gln. In another aspect, the variant comprises or consists of the substitution N175S,G,Q of the full-length polypeptide of SEQ ID NO: 2.

In another aspect, the variant comprises or consists of a substitution at a position corresponding to position 183. In another aspect, the amino acid at a position corresponding to position 183 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile. In another aspect, the variant comprises or consists of the substitution F183I of the full-length polypeptide of SEQ ID NO: 2.

In another aspect, the variant comprises or consists of a substitution at a position corresponding to position 254. In another aspect, the amino acid at a position corresponding to position 254 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu. In another aspect, the variant comprises or consists of the substitution A254E of the full-length polypeptide of SEQ ID NO: 2.

In another aspect, the variant comprises or consists of a substitution at a position corresponding to position 280. In another aspect, the amino acid at a position corresponding to position 280 is substituted with Ala, Arg, Asn, Asp, Cys, Gln, Glu, Gly, His, Ile, Leu, Lys, Met, Phe, Pro, Ser, Thr, Trp, Tyr, or Val, preferably with Gly or Glu. In another aspect, the variant comprises or consists of the substitution S280G,E of the full-length polypeptide of SEQ ID NO: 2.

In each of the aspects above, the variant comprises or consists of one or more substitutions described above at positions corresponding to the full-length polypeptide of SEQ ID NO: 2 in other xyloglucan endotransglycosylases as parents.

In each of the aspects below, the variant comprises or consists of one or more substitutions described below at positions corresponding to the full-length polypeptide of SEQ ID NO: 2 in other xyloglucan endotransglycosylases or at positions of the full-length polypeptide of SEQ ID NO: 2.

In another aspect, the variant comprises or consists of one or more (e.g., several) substitutions selected from the group consisting of I10A; P30E; A40G; S51T; I53A,V; Y605; T99E,N; E102G; Q117E; K103R; R136W; Y157H; Y162C; N175S,G,Q; F183I; A254E; and S280G,E.

In another aspect, the variant comprises or consists of the substitutions A40G+N175S of the full-length polypeptide of SEQ ID NO: 2.

In another aspect, the variant comprises or consists of the substitutions A40G+F183I of the full-length polypeptide of SEQ ID NO: 2.

In another aspect, the variant comprises or consists of the substitutions N175G+S280G of the full-length polypeptide of SEQ ID NO: 2.

In another aspect, the variant comprises or consists of the substitutions A40G+I53A+N175S of the full-length polypeptide of SEQ ID NO: 2.

In another aspect, the variant comprises or consists of the substitutions A40G+N175S+F183I of the full-length polypeptide of SEQ ID NO: 2.

In another aspect, the variant comprises or consists of the substitutions I10A+I53A+E102G of the full-length polypeptide of SEQ ID NO: 2.

In another aspect, the variant comprises or consists of the substitutions A40G+E102G+Q117E of the full-length polypeptide of SEQ ID NO: 2.

In another aspect, the variant comprises or consists of the substitutions N175Q+A254E+S280E of the full-length polypeptide of SEQ ID NO: 2.

In another aspect, the variant comprises or consists of the substitutions P30 E+S51T+Y60S+T99N of the full-length polypeptide of SEQ ID NO: 2.

In another aspect, the variant comprises or consists of the substitutions A40G+T99E+E102G+K130R of the full-length polypeptide of SEQ ID NO: 2.

In another aspect, the variant comprises or consists of the substitutions I53V+R136W+Y157H+Y162C+N175S of the full-length polypeptide of SEQ ID NO: 2.

The variants may further comprise one or more additional alterations, e.g., substitutions, insertions, or deletions at one or more (e.g., several) other positions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R.L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for xyloglucan endotransglycosylase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide. Essential active site amino acids in the sequence of amino acids 21 to 292 of SEQ ID NO: 2 are located at positions 105, 107, and 109.

The present invention also relates to methods of increasing the expression yield of a xyloglucan endotransglycosylase, comprising introducing into a parent xyloglucan endotransglycosylase a substitution at one or more positions corresponding to positions 10, 30, 40, 51, 53, 60, 99, 102, 117, 130, 136, 157, 162, 175, 183, 254, and 280 of the full-length polypeptide of SEQ ID NO: 2 to produce a variant, wherein the expression yield of the variant is increased relative to the parent; and optionally recovering the variant.

In an embodiment, the variant has an increased expression yield compared to the parent enzyme of at least 1.05, at least 1.10, at least 1.20, at least 1.30, at least 1.40, at least 1.50, at least 1.60, at least 1.70, at least 1.80, at least 1.90, at least 2, at least 2.25, at least 2.50, at least 2.75, at least 3.00, at least 3.25, at least 3.50, at least 3.75, at least 4, at least 4.25, at least 4.50, at least 4.75, at least 5, at least 6, at least 7, at least 8, at least 9, or at least 10 fold.

### Parent Xyloglucan Endotransglycosylases

The parent xyloglucan endotransglycosylase may be (a) a polypeptide having at least 60% sequence identity to the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50; (b) a polypeptide encoded by a polynucleotide that hybridizes under at least low stringency conditions with (i) the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, or 49, or (ii) the full-length complement of (i); or (c) a polypeptide encoded by a polynucleotide having at least 60% sequence identity to the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, or 49.

In one embodiment, the parent has a sequence identity to the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have xyloglucan endotransglycosylase activity. In another embodiment, the amino acid sequence of the parent differs by up to 10 amino acids, e.g., 1, 2, 3, 4, 5, 6, 7, 8, 9, or 10, from the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50.

In another embodiment, the parent comprises or consists of the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, or 50. In another embodiment, the parent comprises or consists of the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50.

In another embodiment, the parent is a fragment of the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50 containing at least 85%, e.g., at least 90% and at least 95% of the amino acid residues of the parent.

In another embodiment, the parent is an allelic variant of the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50.

In another embodiment, the parent is encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, or 49, or (ii) the full-length complement of (i) (Sambrook et al., 1989, Molecular Cloning, A Laboratory Manual, 2d edition, Cold Spring Harbor, New York).

The polynucleotide of SEQ ID NO: 1, 3, 5, 7, 9, 11, 13, 15, 17, 19, 21, 23, 25, 27, 29, 31, 33, 35, 37, 39, 41, 43, 45, 47, or 49, or a subsequence thereof, as well as the polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50, or a fragment thereof, may be used to design nucleic acid probes to identify and clone DNA encoding a parent from different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other genera or species may be screened for DNA that hybridizes with the probes described above and encodes a parent. Genomic or other DNA may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1, 3, 5, 7, 9, 11, 13, 15, 17, 19, 21, 23, 25, 27, 29, 31, 33, 35, 37, 39, 41, 43, 45, 47, or 49 or a subsequence thereof, the carrier material is used in a Southern blot.

For purposes of the present invention, hybridization indicates that the polynucleotide hybridizes to a labeled nucleic acid probe corresponding to (i) SEQ ID NO: 1, 3, 5, 7, 9, 11, 13, 15, 17, 19, 21, 23, 25, 27, 29, 31, 33, 35, 37, 39, 41, 43, 45, 47, or 49; (ii) the mature polypeptide coding sequence thereof; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, or 49. In another aspect, the nucleic acid probe is a polynucleotide that encodes the polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50; the mature polypeptide thereof; or a fragment thereof. In another aspect, the nucleic acid probe is SEQ ID NO: 1, 3, 5, 7, 9, 11, 13, 15, 17, 19, 21, 23, 25, 27, 29, 31, 33, 35, 37, 39, 41, 43, 45, 47, or 49.

In another embodiment, the parent is encoded by a polynucleotide having a sequence identity to the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, or 49 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

The parent may be a hybrid polypeptide (chimera) in which a region of the parent is replaced with a region of another polypeptide.

The parent may be a fusion polypeptide or cleavable fusion polypeptide in which another polypeptide is fused at the N-terminus or the C-terminus of the parent. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from plants of any genus. For purposes of the present invention, the term “obtained from” as used herein in connection with a given source shall mean that the parent encoded by a polynucleotide is produced by the source. In one aspect, the parent is secreted extracellularly.

The parent may be any plant xyloglucan endotransglycosylase. In one embodiment, the parent is obtained from cotyledons from the family Fabaceae (synonyms: Leguminosae and Papilionaceae), preferably genera Phaseolus, in particular, Phaseolus aureus. Preferred monocotyledons are non-graminaceous monocotyledons and liliaceous monocotyledons. Xyloglucan endotransglycosylase can also be extracted from moss and liverwort, as described in Fry et al., 1992, Biochem. J. 282: 821-828. For example, the xyloglucan endotransglycosylase may be obtained from cotyledons, i.e., a dicotyledon or a monocotyledon, in particular a dicotyledon selected from the group consisting of cauliflowers, soy beans, tomatoes, potatoes, rapes, sunflowers, cotton, and tobacco, or a monocotyledon selected from the group consisting of wheat, rice, corn, and sugar cane. See, for example, WO 2003/033813 and WO 97/023683.

In another embodiment, the parent is obtained from species of Arabidopsis, Carica, Cucumis, Daucus, Festuca, Glycine, Hordeum, Lycopersicon, Medicago, Oryza, Populus, Sagittaria, Sorghum, Vigna, or Zea.

In another embodiment, the parent is obtained from Arabidopsis thaliana, Carica papaya, Cucumis sativus, Daucus carota, Festuca pratensis, Glycine max, Hordeum vulgare, Lycopersicon esculentum, Medicago truncatula, Oryza sativa, Populus tremula, Sagittaria pygmaea, Sorghum bicolor, Vigna angularis, or Zea mays.

In another embodiment, the parent is an Arabidopsis thaliana (GENESEQP:AOE11231, GENESEQP:AOE93420, GENESEQP: BAL03414, GENESEQP:BAL03622, or GENESEQP:AWK95154); Carica papaya (GENESEQP:AZR75725); Cucumis sativus (GENESEQP:AZV66490); Daucus carota (GENESEQP:AZV66139); Festuca pratensis (GENESEQP:AZR80321); Glycine max (GENESEQP:AWK95154 or GENESEQP:AYF92062); Hordeum vulgare (GENESEQP:AZR85056, GENESEQP:AQY12558, GENESEQP:AQY12559, or GENESEQP:AWK95180); Lycopersicon esculentum (GENESEQP:ATZ45232); Medicago truncatula (GENESEQP:ATZ48025); Oryza sativa (GENESEQP:ATZ42485, GENESEQP:ATZ57524, or GENESEQP:AZR76430); Populus tremula (GENESEQP:AWK95036); Sagittaria pygmaea (GENESEQP:AZV66468); Sorghum bicolor (GENESEQP:BA079623 or GENESEQP:BA079007); Vigna angularis (GENESEQP:ATZ61320); or Zea mays (GENESEQP:AWK94916) xyloglucan endotransglycosylase, wherein the accession numbers are incorporated herein in their entirety.

A polynucleotide encoding a parent may be obtained by screening a genomic DNA or cDNA library or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989, supra).

### Preparation of Variants

The present invention also relates to methods for obtaining a xyloglucan endotransglycosylase variant, comprising: (a) introducing into a parent xyloglucan endotransglycosylase a substitution at one or more (e.g., several) positions corresponding to positions 10, 30, 40, 51, 53, 60, 99, 102, 117, 130, 136, 157, 162, 175, 183, 254, and 280 of the full-length polypeptide of SEQ ID NO: 2, wherein the variant has xyloglucan endotransglycosylase activity; and optionally (b) recovering the variant.

The variants can be prepared using any mutagenesis procedure known in the art, such as site-directed mutagenesis, site-saturation mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Site-saturation mutagenesis systematically replaces a polypeptide coding sequence with sequences encoding all 19 amino acids at one or more (e.g., several) specific positions (Parikh and Matsumura, 2005, J. Mol. Biol. 352: 621-628).

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

### Polynucleotides

The present invention also relates to isolated polynucleotides encoding variants of the present invention.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide recognized by a host cell for expression of a polynucleotide encoding a variant of the present invention. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the variant. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a leader, a nontranslated region of an mRNA that is important for translation by the host cell. The leader is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of the variant and the signal peptide sequence is positioned next to the N-terminus of the propeptide sequence.

It may also be desirable to add regulatory sequences that regulate expression of the variant relative to the growth of the host cell. Examples of regulatory sequences are those that cause expression of the gene to be turned on or off in response to a chemical or physical stimulus, including the presence of a regulatory compound. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked to the regulatory sequence.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors are well known to one skilled in the art (see, e.g., Sambrook et al., 1989, supra).

### Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M.I., editors, Guide to Yeast Genetics and Molecular Biology, Methods in Enzymology, Volume 194, pp. 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant, comprising (a) cultivating a recombinant host cell of the present invention under conditions conducive for production of the variant; and optionally (b) recovering the variant.

The host cells are cultivated in a nutrient medium suitable for production of the variant using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variants may be detected using methods known in the art that are specific for xyloglucan endotransglycosylases. These detection methods include, but are not limited to, use of specific antibodies, formation of an enzyme product, or disappearance of an enzyme substrate. For example, an enzyme assay may be used to determine the activity of the variant, as described herein.

The variant may be recovered using methods known in the art. For example, the variant may be recovered from the nutrient medium by conventional procedures including, but not limited to, collection, centrifugation, filtration, extraction, spray-drying, evaporation, or precipitation. In one aspect, the whole fermentation broth is recovered.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

### Fermentation Broth Formulations or Cell Compositions

The present invention also relates to a fermentation broth formulation or a cell composition comprising a variant of the present invention. The fermentation broth product further comprises additional ingredients used in the fermentation process, such as, for example, cells (including, the host cells containing the gene encoding the variant of the present invention which are used to produce the variant of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In another embodiment, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The fermentation broth formulations or cell compositions may further comprise multiple enzymatic activities, such as one or more (e.g., several) enzymes selected from the group consisting of a hydrolase, an isomerase, a ligase, a lyase, an oxidoreductase, and a transferase.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The present invention also relates to compositions comprising a variant of the present invention. Preferably, the compositions are enriched in such a variant. The term “enriched” indicates that the xyloglucan endotransglycosylase activity of the composition has been increased, e.g., with an enrichment factor of at least 1.1.

The compositions may comprise a variant of the present invention as the major enzymatic component, e.g., a mono-component composition. Alternatively, the compositions may comprise multiple enzymatic activities, such as one or more (e.g., several) enzymes selected from the group consisting of a hydrolase, an isomerase, a ligase, a lyase, an oxidoreductase, and a transferase.

The compositions may be prepared in accordance with methods known in the art and may be in the form of a liquid or a dry composition. The compositions may be stabilized in accordance with methods known in the art.

Examples are given below of preferred uses of the compositions of the present invention. The dosage of the composition and other conditions under which the composition is used may be determined on the basis of methods known in the art.

### Uses

The variants of the present invention can be used in a process for providing a cellulosic material, such as a fabric or a paper and pulp product, with improved strength and/or shape-retention and/or anti-wrinkling properties according to WO 97/23683.

The variants of the present invention can also be used in laundry and/or fabric and/or color care compositions to refurbish and/or restore improved tensile strength, enhanced anti-wrinkle, anti-bobbling and anti-shrinkage properties to cellulosic fabrics according to WO 2001/07556.

The present invention is further described by the following examples that should not be construed as limiting the scope of the invention.

### Examples

Media and Solutions

LB plates were composed of 10 g of tryptone, 5 g of yeast extract, 5 g of NaCl, 15 g of bacteriological agar, and deionized water to 1 liter.

LB medium was composed of 10 g of tryptone, 5 g of yeast extract, 5 g of NaCl, and deionized water to 1 liter.

Minimal medium agar plates were composed of 342.3 g of sucrose, 10 g of glucose, 4 g of MgSO₄.7H₂O, 6 g of NaNO₃, 0.52 g of KCl, 1.52 g of KH₂PO₄, 0.04 mg of Na₂B₄O₇.10H₂O, 0.4 mg of CuSO₄.5H₂O, 1.2 mg of FeSO₄.7H₂O, 0.7 mg of MnSO₄.2H₂O, 0.8 mg of Na₂MoO₄.2H₂O, 10 mg of ZnSO₄.7H₂O, 500 mg of citric acid, 4 mg of d-biotin, 20 g of Noble agar, and deionized water to 1 liter.

Synthetic Defined medium lacking uridine was composed of 18 mg of adenine hemisulfate, 76 mg of alanine, 76 mg of arginine hydrochloride, 76 mg of asparagine monohydrate, 76 mg of aspartic acid, 76 mg of cysteine hydrochloride monohydrate, 76 mg of glutamic acid monosodium salt, 76 mg of glutamine, 76 mg of glycine, 76 mg of histidine, myo-76 mg of inositol, 76 mg of isoleucine, 380 mg of leucine, 76 mg of lysine monohydrochloride, 76 mg of methionine, 8 mg of p-aminobenzoic acid potassium salt, 76 mg of phenylalanine, 76 mg of proline, 76 mg of serine, 76 mg of threonine, 76 mg of tryptophan, 76 mg of tyrosine disodium salt, 76 mg of valine, and deionized water to 1 liter.

YP+2% glucose medium was composed of 10 g of yeast extract, 20 g of peptone, 20 g of glucose, and deionized water to 1 liter.

YP+2% maltodextrin medium was composed of 10 g of yeast extract, 20 g of peptone, 20 g of maltodextrin, and deionized water to 1 liter.

2×YT+ampicillin plates were composed of 16 g of tryptone, 10 g of yeast extract, 5 g of sodium chloride, 15 g of Bacto agar, and deionized water to 1 liter. One ml of a 100 mg/ml solution of ampicillin was added after the autoclaved medium was tempered to 55° C.

TAE buffer was composed of 4.84 g of Tris Base, 1.14 ml of glacial acetic acid, 2 ml of 0.5 M EDTA pH 8.0, and deionized water to 1 liter.

TBE buffer was composed of 10.8 g of Tris Base, 5.5 g boric acid, 4 ml of 0.5 M EDTA pH 8.0, and deionized water to 1 liter.

### Example 1: Iodine Colorimetric Assay to Determine Xyloglucan Endotransglycosylase Activity

Xyloglucan endotransglycosylase activity was assayed using a modified version of an iodine colorimetric assay described by Sulova et al., 1995, Analytical Biochemistry 229: 80-85. For each reaction, 5 μl of tamarind seed xyloglucan (Megazyme International, Bray, UK) (5 mg/ml in water) were combined with 20 μl of xyloglucan oligomers (Megazyme International, Bray, UK) (5 mg/ml in water) and 10 μl of 400 mM sodium citrate pH 5.5, and dispensed into 96 well plates. Reactions were initiated by the addition of 5 μl of liquid culture broth to each well, and plates were incubated at 37° C. for 10 minutes. Reactions were quenched by the addition of 200 μl of a solution composed of 14% (w/v) Na₂SO₄, 0.2% KI, 0.1 M HCl, and 0.5% I₂, and incubated in the dark for 30 minutes prior to measuring the absorbance at 620 nm in a SPECTRAMAX® M5 spectrophotometer (Molecular Devices, Sunnyvale, Calif., USA).

### Example 2: Generation of Fluorescein Isothiocyanate-Labeled Xyloglucan

Fluorescein isothiocyanate-labeled xyloglucan oligomers (FITC-XGOs) were generated by reductive amination of the reducing ends of xyloglucan oligomers according to the procedure described by Zhou et al., 2006, Biocatalysis and Biotransformation 24: 107-120), followed by conjugation of the amino groups of the XGOs to fluorescein isothiocyanate isomer I (Sigma Aldrich, St. Louis, Mo., USA) in 100 mM sodium bicarbonate pH 9.0 for 24 hours at room temperature. Conjugation reaction products were concentrated to dryness in vacuo, dissolved in 0.5 ml of deionized water, and purified by silica gel chromatography, eluting with a gradient from 100:0:0.04 to 70:30:1 acetonitrile:water:acetic acid as mobile phase. Purity and product identity were confirmed by evaporating the buffer, dissolving in D₂O (Sigma Aldrich, St. Louis, Mo., USA), and analysis by ¹H NMR using a Varian 400 MHz MercuryVx (Agilent, Santa Clara, Calif., USA). Dried FITC-XGOs were stored at −20° C. in the dark and desiccated during thaw.

One mg of FITC-XGOs was incubated with 1 mg of tamarind seed xyloglucan (Megazyme, Bray, United Kingdom) and 18 mg of VaXET16 per ml of 20 mM sodium citrate pH 5.0 in 200 μl reactions for at least 30 minutes Sample mixtures were pooled and precipitated by addition of ice cold ethanol to 80% (v/v) final concentration and incubation at 4° C. overnight. Precipitated fluorescein isothiocyanate-labeled xyloglucan (FITC-XG) was recovered by centrifugation at 3000 rpm using a LEGEND™ RT Plus centrifuge (Thermo Scientific, Waltham, Mass., USA), decanting off the ethanol, and drying at room temperature for 24 hours. FITC-XG was dissolved in a minimum volume of deionized water until dissolved and stored at −20° C. Frozen FITC-XG was thawed and lyophilized overnight. The lyophilized powder was dissolved in 500 μl of deionized water and quantified by absorbance at 488 nm.

A large scale batch of FITC-XG was prepared in the following manner. A 7.9 mg per ml solution of FITC-XGOs was prepared in deionized water. Forty ml of 10 mg of tamarind seed xyloglucan (Megazyme, Bray, UK) per ml of deionized water, 452 ml of 7.9 mg of FITC-XGOs per ml of deionized water, 2 ml of 400 mM sodium citrate pH 5.5, and 1.2 ml of 1.4 mg of VaXET16 per ml of 20 mM sodium citrate pH 5.5 were mixed thoroughly and incubated overnight at room temperature. Following overnight incubation, FITC-XG was precipitated by addition of ice cold ethanol to a final volume of 110 ml, mixed thoroughly, and incubated at 4° C. overnight. The precipitated FITC-XG was washed with water and then transferred to Erlenmeyer bulbs. Residual water and ethanol were removed by evaporation using an EZ-2 Elite evaporator (SP Scientific/Genevac, Stone Ridge, N.Y., USA) for 4 hours. Dried samples were dissolved in water, and the volume was adjusted to 48 ml with deionized water to generate a final FITC-XG concentration of 5 mg per ml with an expected average molecular weight of 100 kDa.

### Example 3: Fluorescence Polarization Assay to Determine Xyloglucan Endotransglycosylation Activity

Xyloglucan endotransglycosylation activity was determined using the following assay. Reactions of 200 μl containing 1 mg of tamarind seed xyloglucan per ml, 0.01 mg/ml FITC-XGOs prepared as described in Example 2, and 10 μl of appropriately diluted XET were incubated for 10 minutes at 25° C. in 20 mM sodium citrate pH 5.5 in opaque 96-well microtiter plates. Fluorescence polarization was monitored continuously over this time period, using a SPECTRAMAX® M5 microplate reader (Molecular Devices, Sunnyvale, Calif., USA) in top-read orientation with an excitation wavelength of 490 nm, an emission wavelength of 520 nm, a 495 cutoff filter in the excitation path, high precision (100 reads), and medium photomultiplier tube sensitivity. XET-dependent incorporation of fluorescent XGOs into non-fluorescent XG results in increasing fluorescence polarization over time. The slope of the linear regions of the polarization time progress curves was used to determine the activity.

### Example 4: Construction of Plasmid pMMar27

Plasmid pMMar27 was constructed for expression of the T. terrestris Cel6A cellobiohydrolase II in yeast. The plasmid was generated from a lineage of yeast expression vectors: plasmid pMMar27 was constructed from plasmid pBM175b; plasmid pBM175b was constructed from plasmid pBM143b (WO 2008/008950) and plasmid pJLin201; and plasmid pJLin201 was constructed from pBM143b.

Plasmid pJLin201 is identical to pBM143b except an Xba I site immediately downstream of a Thermomyces lanuginosus lipase variant gene in pBM143b was mutated to a unique Nhe I site. A QUIKCHANGE® II XL Site-Directed Mutagenesis Kit (Stratagene, La Jolla, Calif., USA) was used to change the Xba I sequence (TCTAGA) to a Nhe I sequence (gCTAGc) in pBM143b. Primers employed to mutate the site are shown below.

Primer 999551 (sense): 5′-ACATGTCTTTGATAAgCTAGcGGGCCGCATCATGTA-3′ (SEQ ID NO: 52)
Primer 999552 (antisense): 5′-TACATGATGCGGCCCgCTAGcTTATCAAAGACATGT-3′ (SEQ ID NO: 53) Lower case represents mutated nucleotides.

The PCR was composed of 125 ng of each primer above, 20 ng of pBM143b, 1× QUIKCHANGE® Reaction Buffer (Stratagene, La Jolla, Calif., USA), 3 μl of QUIKSOLUTION® (Stratagene, La Jolla, Calif., USA), 1 μl of dNTP mix, and 1 μl of a 2.5 units/ml Pfu Ultra HF DNA polymerase (Stratagene, La Jolla, Calif., USA) in a final volume of 50 μl. The reaction was performed using an EPPENDORF® MASTERCYCLER® (Eppendorf AG, Hamburg, Germany) programmed for 1 cycle at 95° C. for 1 minute; 18 cycles each at 95° C. for 50 seconds, 60° C. for 50 seconds, and 68° C. for 6 minutes and 6 seconds; and 1 cycle at 68° C. for 7 minutes. After the PCR reaction, the tube was placed on ice for 2 minutes. One microliter of Dpn I (Promega, Fitchburg, Wis., USA) was directly added to the PCR and incubated at 37° C. for 1 hour. A 2 μl volume of the Dpn I digested reaction was used to transform E. coli XL10 GOLD® Ultracompetent Cells (Stratagene, La Jolla, Calif., USA) according to the manufacturer's instructions. E. coli transformants were selected on 2×YT plus ampicillin plates. Plasmid DNA was isolated from several of the transformants using a BIOROBOT® 9600 (QIAGEN Inc., Valencia, Calif., USA). One plasmid with the desired Nhe I change was confirmed by restriction digestion and sequencing analysis and designated plasmid pJLin201. To eliminate possible PCR errors introduced by site-directed-mutagenesis, plasmid pBM175b was constructed by cloning the Nhe I site containing fragment back into plasmid pBM143b. Briefly, plasmid pJLin201 was digested with Nde I and Mlu I and the resulting fragment was cloned into pBM143b previously digested with the same enzymes using a Rapid Ligation Kit (Roche Diagnostics Corporation, Indianapolis, Ind., USA). Briefly, 7 μl of the Nde I/Mlu I digested pJLin201 fragment and 1 μl of the digested pBM143b were mixed with 2 μl of 5×DNA dilution buffer (Roche Diagnostics Corporation, Indianapolis, Ind., USA), 10 μl of 2× T4 DNA Ligation buffer (Roche Diagnostics Corporation, Indianapolis, Ind., USA), and 1 μl of T4 DNA ligase (Roche Diagnostics Corporation, Indianapolis, Ind., USA) and incubated for 15 minutes at room temperature. Two microliters of the ligation were transformed into XL1-Blue Subcloning-Grade Competent Cells (Stratagene, La Jolla, Calif., USA) cells and spread onto 2×YT plus ampicillin plates. Plasmid DNA was purified from several transformants using a BIOROBOT® 9600 and analyzed by DNA sequencing using a 3130XL Genetic Analyzer (Applied Biosystems, Foster City, Calif., USA) to identify a plasmid containing the desired A. nidulans pyrG insert. One plasmid with the expected DNA sequence was designated pBM175b.

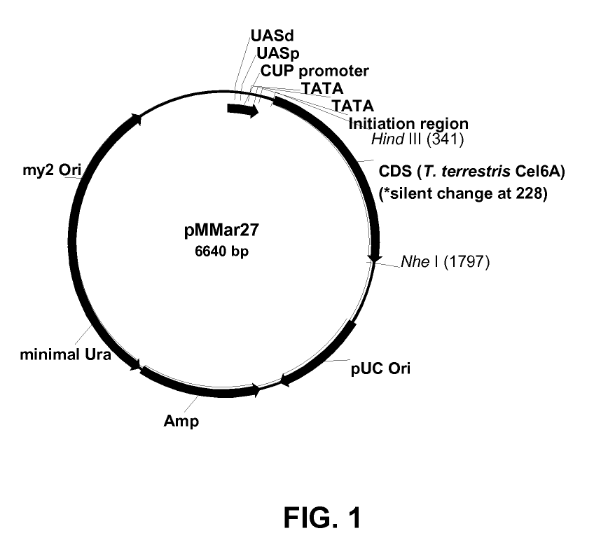

Plasmid pMMar27 was constructed from pBM175b and an amplified gene of T. terrestris Cel6A cellobiohydrolase II with overhangs designed for insertion into digested pBM175b. Plasmid pBM175b containing the Thermomyces lanuginosus lipase variant gene under control of the CUP I promoter contains unique Hind III and Nhe I sites to remove the lipase gene. Plasmid pBM175b was digested with these restriction enzymes to remove the lipase gene. After digestion, the empty vector was isolated by 1.0% agarose gel electrophoresis using TBE buffer where an approximately 5,215 bp fragment was excised from the gel and extracted using a QIAQUICK® Gel Extraction Kit (QIAGEN Inc., Valencia, Calif., USA). The ligation reaction (20 μl) was composed of 1× IN-FUSION® Buffer (BD Biosciences, Palo Alto, Calif., USA), 1×BSA (BD Biosciences, Palo Alto, Calif., USA), 1 μl of IN-FUSION® enzyme (diluted 1:10) (BD Biosciences, Palo Alto, Calif., USA), 99 ng of pBM175b digested with Hind III and Nhe I, and 36 ng of the purified T. terrestris Cel6A cellobiohydrolase II PCR product. The reaction was incubated at room temperature for 30 minutes. A 2 μl volume of the IN-FUSION® reaction was transformed into E. coli XL10-GOLD® Ultracompetent Cells (Stratagene, La Jolla, Calif., USA). Transformants were selected on LB plates supplemented with 100 μg of ampicillin per ml. A colony was picked that contained the T. terrestris Cel6A inserted into the pBM175b vector in place of the lipase gene, resulting in pMMar27 (FIG. 1). The plasmid chosen contained a PCR error at position 228 from the start codon, TCT instead of TCC, but resulted in a silent change in the T. terrestris Cel6A cellobiohydrolase II.

### Example 5: Construction of pEvFz1 Expression Vector

Expression vector pEvFz1 was constructed by modifying pBM120a (U.S. Pat. No. 8,263,824) to comprise the NA2/NA2-tpi promoter, A. niger amyloglucosidase terminator sequence (AMG terminator), and Aspergillus nidulans orotidine-5′-phosphate decarboxylase gene (pyrG) as a selectable marker.

Plasmid pEvFz1 was generated by cloning the A. nidulans pyrG gene from pAILo2 (WO 2004/099228) into pBM120a. Plasmids pBM120a and pAILo2 were digested with Nsi I overnight at 37° C. The resulting 4176 bp linearized pBM120a vector fragment and the 1479 bp pyrG gene insert from pAILo2 were each purified by 0.7% agarose gel electrophoresis using TAE buffer, excised from the gel, and extracted using a QIAQUICK® Gel Extraction Kit.

The 1479 bp pyrG gene insert was ligated to the Nsi I digested pBM120a fragment using a QUICK LIGATION™ Kit (New England Biolabs, Beverly, Mass., USA). The ligation reaction was composed of 1× QUICK LIGATION™ Reaction Buffer (New England Biolabs, Beverly, Mass., USA), 50 ng of Nsi I digested pBM120a vector, 54 ng of the 1479 bp Nsi I digested pyrG gene insert, and 1 μl of T4 DNA Ligase in a total volume of 20 μl. The ligation mixture was incubated at 37° C. for 15 minutes followed at 50° C. for 15 minutes and then placed on ice.

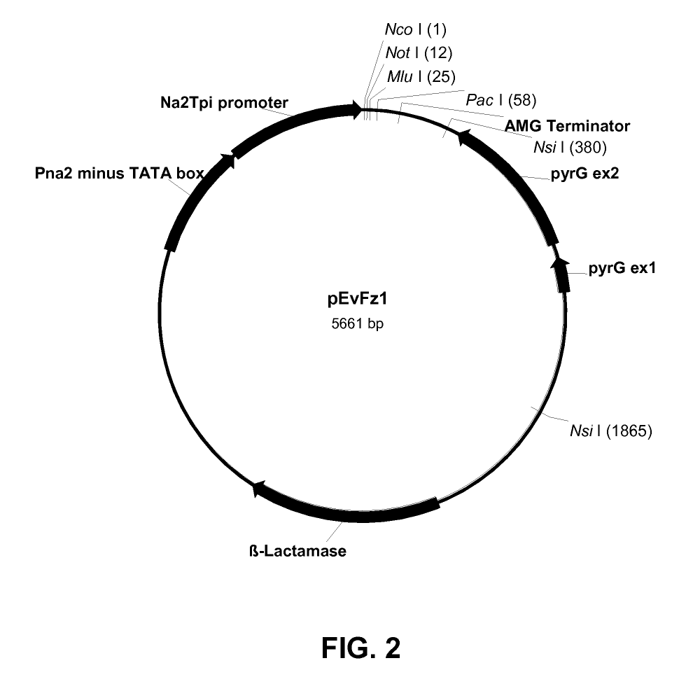

One μl of the ligation mixture was transformed into ONE SHOT® TOP10 chemically competent Escherichia coli cells (Invitrogen, Carlsbad, Calif., USA). Transformants were selected on 2×YT plus ampicillin plates. Plasmid DNA was purified from several transformants using a BIOROBOT® 9600 and analyzed by DNA sequencing using a 3130XL Genetic Analyzer to identify a plasmid containing the desired A. nidulans pyrG insert. One plasmid with the expected DNA sequence was designated pEvFz1 (FIG. 2).

### Example 6: Construction of the Plasmid pDLHD0006 as a Yeast/E. coli/A. Oryzae Shuttle Vector

Plasmid pDLHD0006 was constructed as a base vector to enable A. oryzae expression cassette library building using yeast recombinational cloning. Plasmid pDLHD0006 was generated by combining three DNA fragments using yeast recombinational cloning (Table I): Fragment 1 containing the E. coli pUC origin of replication, E. coli beta-lactamase (ampR) selectable marker, URA3 yeast selectable marker, and yeast 2 micron origin of replication from pMMar27 (Example 4); Fragment 2 containing the NA2-tpi promoter (a hybrid of the promoters from the genes encoding Aspergillus niger neutral alpha-amylase and Aspergillus oryzae triose phosphate isomerase, Thermomyces lanuginosus lipase open reading frame (ORF), and Aspergillus niger glucoamylase terminator from pJaL1262 (WO 2013/178674); and Fragment 3 containing the Aspergillus nidulans pyrG selection marker from pEvFz1 (Example 5).

TABLE I

| pDLHD0006 | PCR Contents | PCR Template |
|---|---|---|
| Fragment 1 | E. coli ori/AmpR/URA/2 micron (4.1 kb) | pMMar27 |
| Fragment 2 | NA2-tpi PR/lipase/Tamg (4.5 kb) | pJaL1262 |
| Fragment 3 | pyrG gene from pEvFz1 (1.7 kb) | pEvFz1 |

Fragment 1 was amplified using primers 613017 (sense) and 613018 (antisense) shown below. Primer 613017 was designed to contain a flanking region with sequence homology to Fragment 3 (lower case) and primer 613018 was designed to contain a flanking region with sequence homology to Fragment 2 (lower case) to enable yeast recombinational cloning between the three PCR fragments.

Primer 613017 (sense):
(SEQ ID NO: 54)
ttaatcgccttgcagcacaCCGCTTCCTCGCTCACTGACTC
Primer 613018 (antisense):
(SEQ ID NO: 55)
acaataaccctgataaatgcGGAACAACACTCAACCCTATCTCGGTC

Fragment 1 was amplified by PCR in a reaction composed of 10 ng of plasmid pMMar27, 0.5 μl of PHUSION® DNA Polymerase (New England Biolabs, Inc., Ipswich, Mass., USA), 20 pmol of primer 613017, 20 pmol of primer 613018, 1 μl of 10 mM dNTPs, 10 μl of 5× PHUSION® HF buffer (New England Biolabs, Inc., Ipswich, Mass., USA), and 35.5 μl of water. The reaction was incubated in an EPPENDORF® MASTERCYCLER® thermocycler programmed for 1 cycle at 98° C. for 30 seconds; and 30 cycles each at 98° C. for 10 seconds, 60° C. for 10 seconds, and 72° C. for 1.5 minutes. The resulting 4.1 kb PCR product (Fragment 1) was used directly for yeast recombination with Fragments 2 and 3 below.

Fragment 2 was amplified using primers 613019 (sense) and 613020 (antisense) shown below. Primer 613019 was designed to contain a flanking region of sequence homology to Fragment 1 (lower case) and primer 613020 was designed to contain a flanking region of sequence homology to Fragment 3 (lower case) to enable yeast recombinational cloning between the three PCR fragments.

613019 (sense):
(SEQ ID NO: 56)
agatagggttgagtgttgttccGCATTTATCAGGGTTATTGTCTCATGAG
CGG
613020 (antisense):
(SEQ ID NO: 57)
ttctacacgaaggaaagagGAGGAGAGAGTTGAACCTGGACG

Fragment 2 was amplified by PCR in a reaction composed of 10 ng of plasmid pJaL1262, 0.5 μl of PHUSION® DNA Polymerase, 20 pmol of primer 613019, 20 pmol of primer 613020, 1 μl of 10 mM dNTPs, 10 μl of 5× PHUSION® HF buffer, and 35.5 μl of water. The reaction was incubated in an EPPENDORF® MASTERCYCLER® thermocycler programmed for 1 cycle at 98° C. for 30 seconds; 30 cycles each at 98° C. for 10 seconds, 60° C. for 10 seconds, and 72° C. for 2 minutes; and a 20° C. hold. The resulting 4.5 kb PCR product (Fragment 2) was used directly for yeast recombination with Fragment 1 above and Fragment 3 below.

Fragment 3 was amplified using primers 613022 (sense) and 613021 (antisense) shown below. Primer 613021 was designed to contain a flanking region of sequence homology to Fragment 2 (lower case) and primer 613022 was designed to contain a flanking region of sequence homology to Fragment 1 (lower case) to enable yeast recombinational cloning between the three PCR fragments.

Primer 613022 (sense):
(SEQ ID NO: 58)
aggttcaactctctcctcCTCTTTCCTTCGTGTAGAAGACCAGACAG
Primer 613021 (antisense):
(SEQ ID NO: 59)
tcagtgagcgaggaagcggTGTGCTGCAAGGCGATTAAGTTGG

Fragment 3 was amplified by PCR in a reaction composed of 10 ng of plasmid pEvFz1 (Example 5), 0.5 μl of PHUSION® DNA Polymerase, 20 pmol of primer 613021, 20 pmol of primer 613022, 1 μl of 10 mM dNTPs, 10 μl of 5× PHUSION® HF buffer, and 35.5 μl of water. The reaction was incubated in an EPPENDORF® MASTERCYCLER® thermocycler programmed for 1 cycle at 98° C. for 30 seconds; 30 cycles each at 98° C. for 10 seconds, 60° C. for 10 seconds, and 72° C. for 2 minutes; and a 20° C. hold. The resulting 1.7 kb PCR product (Fragment 3) was used directly for yeast recombination with Fragments 1 and 2 above.

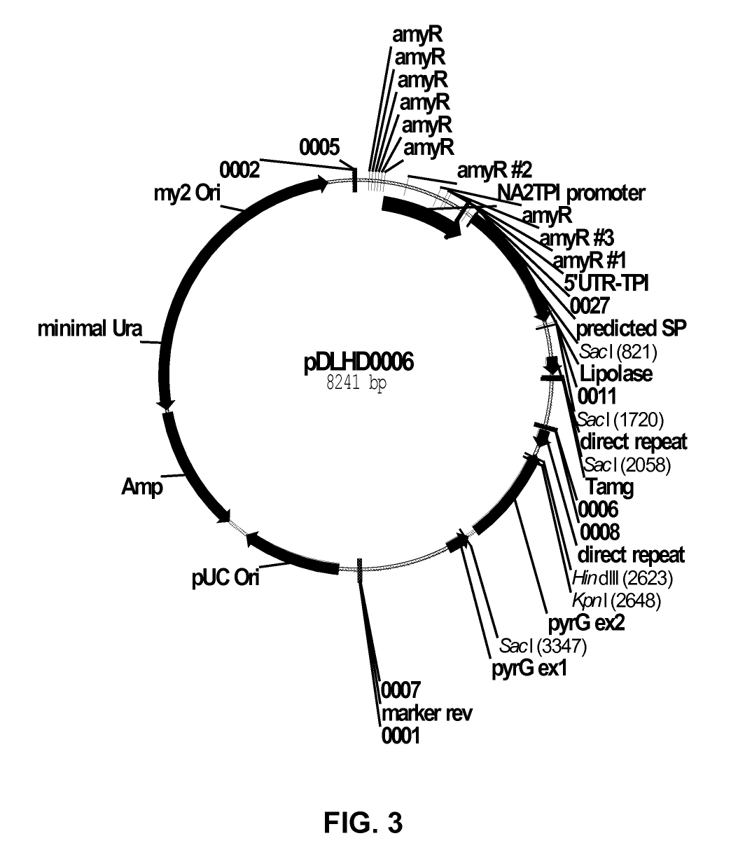

The following procedure was used to combine the three PCR fragments using yeast homology-based recombinational cloning. A 20 μl aliquot of each of the three PCR fragments was combined with 100 μg of single-stranded deoxyribonucleic acid from salmon testes (Sigma-Aldrich, St. Louis, Mo., USA), 100 μl of competent yeast cells of strain YNG318 (Saccharomyces cerevisiae ATCC 208973), and 600 μl of PLATE Buffer (Sigma Aldrich, St. Louis, Mo., USA), and mixed. The reaction was incubated at 30° C. for 30 minutes with shaking at 200 rpm. The reaction was then continued at 42° C. for 15 minutes with no shaking. The cells were pelleted by centrifugation at 5,000×g for 1 minute and the supernatant was discarded. The cell pellet was suspended in 200 μl of autoclaved water and split over two agar plates containing Synthetic Defined medium lacking uridine and incubated at 30° C. for three days. The yeast colonies were isolated from the plate using 1 ml of autoclaved water. The cells were pelleted by centrifugation at 13,000×g for 30 seconds and a 100 μl aliquot of glass beads were added to the tube. The cell and bead mixture was suspended in 250 μl of P1 buffer (QIAGEN Inc., Valencia, Calif., USA) and then vortexed for 1 minute to lyse the cells. The plasmid DNA was purified using a QIAPREP® Spin Miniprep Kit (QIAGEN Inc., Valencia, Calif., USA). A 3 μl aliquot of the plasmid DNA was then transformed into E. coli ONE SHOT® TOP10 electrocompetent cells (Invitrogen, Carlsbad, Calif., USA) according the manufacturer's instructions. Fifty μl of transformed cells were spread onto LB plates supplemented with 100 μg of ampicillin per ml and incubated at 37° C. overnight. Transformants were each picked into 3 ml of LB medium supplemented with 100 μg of ampicillin per ml and grown overnight at 37° C. with shaking at 250 rpm. The plasmid DNA was purified from colonies using a QIAPREP® Spin Miniprep Kit. DNA sequencing with a 3130XL Genetic Analyzer was used to confirm the presence of each of the three fragments in a final plasmid designated pDLHD0006 (FIG. 3).

### Example 7: Construction of pDLH0044 for Expression of the Wild-Type Vigna angularis Xyloglucan Endotransglycosylase 16 (VaXET16) in Aspergillus oryzae

The wild-type VaXET16 cDNA was codon-optimized and synthesized for expression in Aspergillus oryzae. Plasmid pDLHD0044 was constructed to express the codon-optimized VaXET16 gene in multi-copy in Aspergillus oryzae. Plasmid pDLHD0044 was generated by combining two DNA fragments using a GENEART® Seamless Cloning and Assembly Kit (Invitrogen, Carlsbad, Calif., USA): fragment 1 containing the VaXET16 synthetic gene from GENEART® supplied in vector pMA, and flanking sequences with homology to fragment 2, and fragment 2 consisting of an inverse PCR product of vector pDLHD0006 containing the E. coli pUC origin of replication, E. coli beta-lactamase (ampR) selectable marker, URA3 yeast selectable marker, yeast 2 micron origin of replication, the NA2-tpi promoter, the A. niger amyloglucosidase terminator sequence (AMG terminator), and the Aspergillus nidulans orotidine-5′-phosphate decarboxylase gene (pyrG) as a selectable marker.

Fragment 1 was amplified using primer 614603 (sense) and primer 614605 (antisense) shown below. These primers were designed to contain flanking regions of sequence homology to fragment 2 (lower case) for ligation-free cloning.

Primer 614603 (sense):
(SEQ ID NO: 60)
ttcctcaatcctctatatacacaactggccATGGGCTCGTCCCTCTGGAC
Primer 614605 (antisense):
(SEQ ID NO: 61)
agctcgctagagtcgacctaGATGTCCCTATCGCGTGTACACTCG

Fragment 1 was amplified by PCR in a reaction composed of 10 ng of pMA-VaXET16, 0.5 μl of PHUSION® DNA Polymerase, 20 pmol of primer 614603, 20 pmol of primer 614605, 1 μl of 10 mM dNTPs, 10 μl of 5× PHUSION® HF buffer, and 35.5 μl of water. The reaction was incubated in an EPPENDORF® MASTERCYCLER® thermocycler programmed for 1 cycle at 98° C. for 30 seconds; and 30 cycles each at 98° C. for 10 seconds, 60° C. for 10 seconds, and 72° C. for 60 seconds. The resulting 0.9 kb PCR product (fragment 1) was treated with 1 μl of Dpn Ito remove plasmid template DNA. The Dpn I was added directly to the PCR tube, mixed well, and incubated at 37° C. for 60 minutes.

Fragment 2 was amplified using primer 614604 (sense) and primer 613247 (antisense) shown below.

Primer 614604 (sense):
(SEQ ID NO: 62)
taggtcgactctagcgagctcgagatc
Primer 613247 (antisense):
(SEQ ID NO: 63)
catggccagttgtgtatatagaggattgaggaaggaagag

Fragment 2 was amplified by PCR in a reaction composed of 10 ng of pDLHD0006, 0.5 μl of PHUSION® DNA Polymerase, 20 pmol of primer 614604, 20 pmol of primer 613247, 1 μl of 10 mM dNTPs, 10 μl of 5× PHUSION® HF buffer, and 35.5 μl of water. The reaction was incubated in an EPPENDORF® MASTERCYCLER® thermocycler programmed for 1 cycle at 98° C. for 30 seconds; and 30 cycles each at 98° C. for 10 seconds, 60° C. for 10 seconds, and 72° C. for 4 minutes. The resulting 7.3 kb PCR product (fragment 2) was treated with 1 μl of Dpn I to remove plasmid template DNA. The Dpn I was added directly to the PCR tube, mixed well, and incubated at 37° C. for 60 minutes.

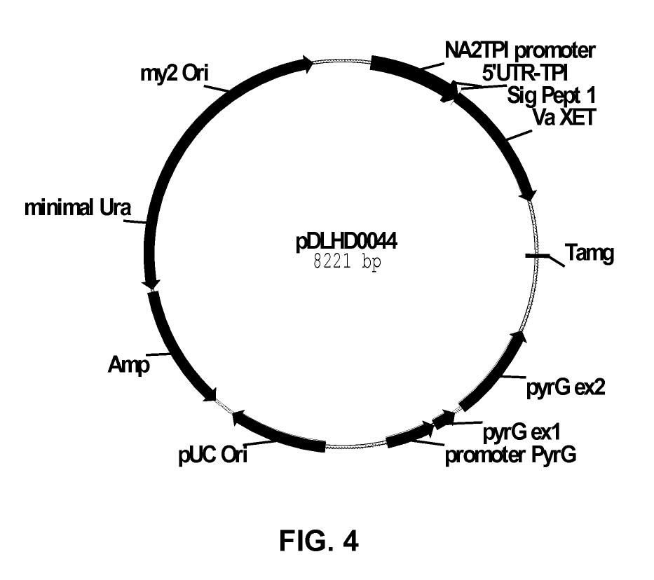

The following procedure was used to combine the two PCR fragments using a GENEART® Seamless Cloning and Assembly Kit. A 5 μl aliquot of each of the PCR fragments was added to a microcentrifuge tube, followed by the addition of 4 μl of water, 4 μl of 5× Reaction Buffer (Invitrogen, Carlsbad, Calif., USA), and 2 μl of 10× Enzyme Mix (Invitrogen, Carlsbad, Calif., USA). The reaction was incubated for 30 minutes at room temperature. A 3 μl aliquot of the reaction mixture was then transformed into E. coli ONE SHOT® TOP10 electrocompetent cells according the manufacturer's instructions. Fifty μl of transformed cells were spread onto 2×YT plates supplemented with 100 μg of ampicillin per ml and incubated at 37° C. overnight. Transformants were each picked into 3 ml of LB medium supplemented with 100 μg of ampicillin per ml and grown overnight at 37° C. with shaking at 250 rpm. The plasmid DNA was purified from colonies using a QIAPREP® Spin Miniprep Kit. DNA sequencing with a 3130XL Genetic Analyzer was used to confirm the presence of each of the three fragments in a final plasmid designated plasmid pDLHD0044 (FIG. 4).

### Example 8: Cloning of the Wild-Type Vigna angularis Xyloglucan Endotransglycosylase 16 (VaXET16) for Expression in Aspergillus oryzae

The codon-optimized wild-type VaXET16 cDNA (Example 7) was cloned into a Saccharomyces cerevisiae/A. oryzae Flp/FRT shuttle vector by yeast recombinational cloning, resulting in vector pDLHD0075.

Expression vector pDLHD0075 was constructed to contain the E. coli pUC origin of replication, E. coli beta-lactamase (ampR) selectable marker, URA3 yeast selectable marker, yeast 2 micron origin of replication, NA2-tpi promoter, codon-optimized VaXET16 open reading frame (ORF; SEQ ID NO: 51 for the DNA sequence and SEQ ID NO: 2 for the deduced amino acid sequence), Aspergillus niger glucoamylase terminator, Aspergillus nidulans pyrG selection marker, Saccharomyces cerevisiae 2 μm flippase ORF between the Aspergillus oryzae TEF1 promoter and Aspergillus oryzae NiaD terminator, and Saccharomyces cerevisiae 2 μm flippase recognition targets FRT-F and FRT-F3.

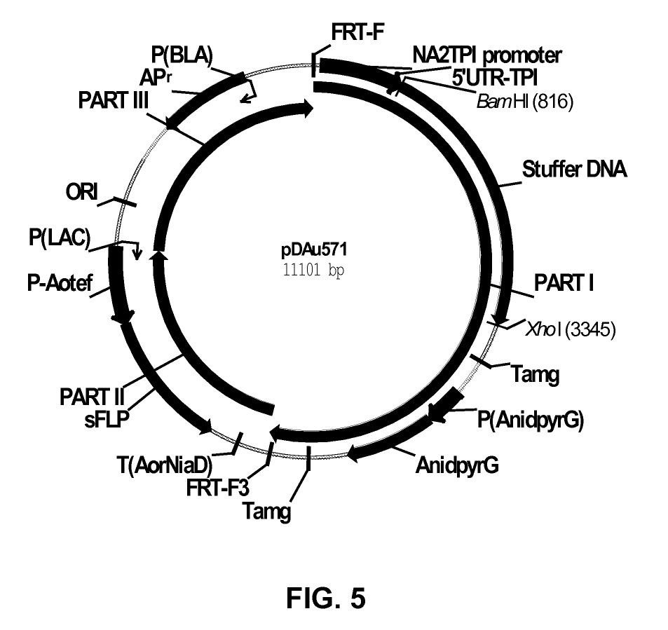

Plasmid pDLHD0075 was generated by combining four DNA fragments using yeast recombinational cloning: Fragment 1 contained the flippase expression cassette, FRT-F3, and AMG terminator from pDau571 (FIG. 5; SEQ ID NO: 64) and flanking sequences with homology to fragments 4 and 2. Fragment 2 contained the E. coli pUC origin of replication, E. coli beta-lactamase (ampR) selectable marker, URA3 yeast selectable marker, yeast 2 micron origin of replication from pDLHD0044, and flanking sequences with homology to fragments 1 and 3. Fragment 3 contained the NA2-tpi promoter, the VaXET16 codon-optimized gene from pDLHD0044, and flanking sequences with homology to fragments 2 and 4. Fragment 4 contained the A. niger amyloglucosidase terminator sequence (AMG terminator) and Aspergillus nidulans orotidine-5′-phosphate decarboxylase gene (pyrG) as a selectable marker from pDau571, and flanking sequences with homology to fragments 3 and 1.

Fragment 1 was amplified using primer 615726 (sense) and primer 615728 (antisense) shown below. These primers were designed to contain flanking regions of sequence homology to fragments 4 and 2, respectively (lower case), for ligation-free cloning between the PCR fragments.

Primer 615726 (sense):
(SEQ ID NO: 65)
accgggaggaaggctggaaaGCTTACGAGAAAAGAGTTGGACTTTGAGGG
Primer 615728 (antisense):
(SEQ ID NO: 66)
tgagcgaggaagcggAAGAGCGCCCAATACGCAAACCGCC

Fragment 1 was amplified by PCR in a reaction composed of 10 ng of pDau571, 0.5 μl of PHUSION® DNA Polymerase, 20 pmol of primer 615726, 20 pmol of primer 615728, 1 μl of 10 mM dNTPs, 10 μl of 5× PHUSION® HF buffer, and 35.5 μl of water. The reaction was incubated in an EPPENDORF® MASTERCYCLER® thermocycler programmed for 1 cycle at 98° C. for 30 seconds; and 30 cycles each at 98° C. for 10 seconds, 60° C. for 10 seconds, and 72° C. for 120 seconds. The resulting 3.3 kb PCR product (fragment 1) was treated with 1 μl of Dpn I to remove plasmid template DNA. The Dpn I was added directly to the PCR tube, mixed well, and incubated at 37° C. for 60 minutes.

Fragment 2 was amplified using primer 615729 (sense) and primer 615731 (antisense) shown below. These primers were designed to contain flanking regions of sequence homology to fragments 1 and 3, respectively (lower case), for ligation-free cloning between the PCR fragments.

Primer 615729 (sense):
(SEQ ID NO: 67)
tgcgtattgggcgctcttCCGCTTCCTCGCTCACTGACTC
Primer 615731 (antisense):
(SEQ ID NO: 68)
tatactttctagagaataggaactcggaataggaacttcaaGGAACAACA
CTCAACCCTATCTCGGTC

Fragment 2 was amplified by PCR in a reaction composed of 10 ng of pDLHD0044, 0.5 μl of PHUSION® DNA Polymerase, 20 pmol of primer 615729, 20 pmol of primer 615731, 1 μl of 10 mM dNTPs, 10 μl of 5× PHUSION® HF buffer, and 35.5 μl of water. The reaction was incubated in an EPPENDORF® MASTERCYCLER® thermocycler programmed for 1 cycle at 98° C. for 30 seconds; and 30 cycles each at 98° C. for 10 seconds, 60° C. for 10 seconds, and 72° C. for 120 seconds. The resulting 4.2 kb PCR product (fragment 2) was treated with 1 μl of Dpn I to remove plasmid template DNA. The Dpn I was added directly to the PCR tube, mixed well, and incubated at 37° C. for 60 minutes.

Fragment 3 was amplified using primer 615730 (sense) and primer 615611 (antisense) shown below. These primers were designed to contain flanking regions of sequence homology to fragments 2 and 4, respectively (lower case), for ligation-free cloning between the PCR fragments.

Primer 615730 (sense):
(SEQ ID NO: 69)
tccgagttcctattctctagaaagtataggaacttcGCATTTATCAGGG
TTATTGTCTCATGAGCGG
Primer 615611 (antisense):
(SEQ ID NO: 70)
tctagatctcgagtcaGATGTCCCTATCGCGTGTACACTCG

Fragment 3 was amplified by PCR in a reaction composed of 10 ng of pDLHD0044, 0.5 μl of PHUSION® DNA Polymerase, 20 pmol of primer 615730, 20 pmol of primer 615611, 1 μl of 10 mM dNTPs, 10 μl of 5× PHUSION® HF buffer, and 35.5 μl of water. The reaction was incubated in an EPPENDORF® MASTERCYCLER® thermocycler programmed for 1 cycle at 98° C. for 30 seconds; and 30 cycles each at 98° C. for 10 seconds, 60° C. for 10 seconds, and 72° C. for 120 seconds. The resulting 1.7 kb PCR product (fragment 3) was treated with 1 μl of Dpn I to remove plasmid template DNA. The Dpn I was added directly to the PCR tube, mixed well, and incubated at 37° C. for 60 minutes.

Fragment 4 was amplified using primer 615610 (sense) and primer 615727 (antisense) shown below. These primers were designed to contain flanking regions of sequence homology to fragments 3 and 1, respectively (lower case), for ligation-free cloning between the PCR fragments.

Primer 615610 (sense):
(SEQ ID NO: 71)
acacgcgatagggacatcTGACTCGAGATCTAGAGGGTGACTGAC
Primer 615727 (antisense):
(SEQ ID NO: 72)
aactcttttctcgtaagcTTTCCAGCCTTCCTCCCGGTAC

Fragment 4 was amplified by PCR in a reaction composed of 10 ng of pDau571, 0.5 μl of PHUSION® DNA Polymerase, 20 pmol of primer 615610, 20 pmol of primer 615727, 1 μl of 10 mM dNTPs, 10 μl of 5× PHUSION® HF buffer, and 35.5 μl of water. The reaction was incubated in an EPPENDORF® MASTERCYCLER® thermocycler programmed for 1 cycle at 98° C. for 30 seconds; and 30 cycles each at 98° C. for 10 seconds, 60° C. for 10 seconds, and 72° C. for 120 seconds. The resulting 1.9 kb PCR product (fragment 4) was treated with 1 μl of Dpn I to remove plasmid template DNA. The Dpn I was added directly to the PCR tube, mixed well, and incubated at 37° C. for 60 minutes.

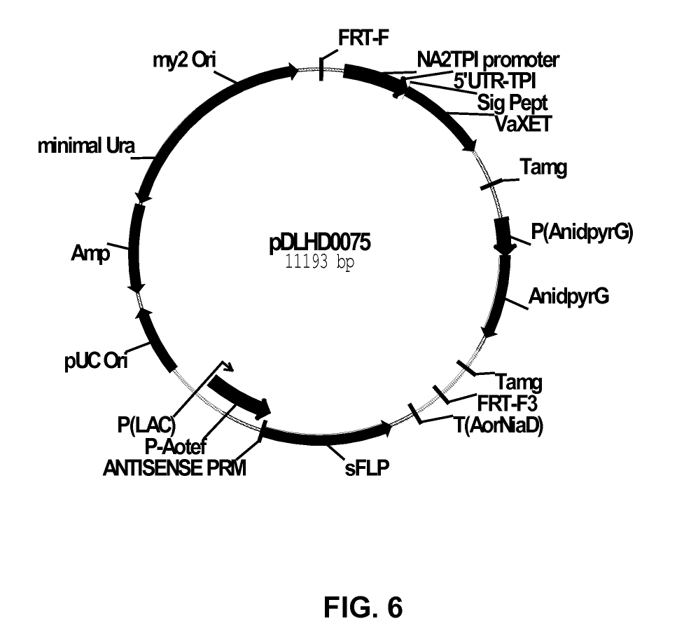

The following procedure was used to combine the four PCR fragments using yeast homology-based recombinational cloning. A 10 μl aliquot of each of the PCR fragments was combined with 100 μg of single-stranded deoxyribonucleic acid from salmon testes (Sigma-Aldrich, St. Louis, Mo., USA), 100 μl of competent yeast cells of strain YNG318 (Saccharomyces cerevisiae ATCC 208973), and 600 μl of PLATE Buffer (Sigma Aldrich, St. Louis, Mo., USA), and mixed. The reaction was incubated at 30° C. for 30 minutes with shaking at 200 rpm. The reaction was then continued at 42° C. for 15 minutes with no shaking. The cells were pelleted by centrifugation at 5,000×g for 1 minute and the supernatant was discarded. The cell pellet was suspended in 200 μl of autoclaved water and split over two agar plates containing Synthetic Defined medium lacking uridine and incubated at 30° C. for three days. The yeast colonies were isolated from the plate using 1 ml of autoclaved water. The cells were pelleted by centrifugation at 13,000×g for 30 seconds and a 100 μl aliquot of glass beads were added to the tube. The cell and bead mixture was suspended in 250 μl of P1 buffer (QIAGEN Inc., Valencia, Calif., USA) and then vortexed for 1 minute to lyse the cells. The plasmid DNA was purified using a QIAPREP® Spin Miniprep Kit. A 3 μl aliquot of the plasmid DNA was then transformed into E. coli ONE SHOT® TOP10 electrocompetent cells according the manufacturer's instructions. Fifty μl of transformed cells were spread onto 2× YT plates supplemented with 100 μg of ampicillin per ml and incubated at 37° C. overnight. Transformants were each picked into 3 ml of LB medium supplemented with 100 μg of ampicillin per ml and grown overnight at 37° C. with shaking at 250 rpm. The plasmid DNA was purified from colonies using a QIAPREP® Spin Miniprep Kit. DNA sequencing with a 3130XL Genetic Analyzer was used to confirm the presence of each of the three fragments in a final plasmid designated plasmid pDLHD0075 (FIG. 6).

### Example 9: Confirmation of Wild-Type Vigna angularis Xyloglucan Endotransglycosylase 16 (VaXET16) Expression in Single-Copy in Aspergillus oryzae Strain JaL1394

Aspergillus oryzae strain JaL1394 (WO 2012/160093) utilizing the Saccharomyces cerevisiae 2 μm plasmid flippase recognition target (FRT) and recombinase (Flp) system to generate a high efficiency single-copy, targeted transformation system was used for screening gene variant libraries. The Flp-FRT system of Saccharomyces cerevisiae is a site-specific recombination system which can be used to insert a DNA of interest into a known location in the genome of a host organism of interest. The Aspergillus oryzae strain JaL1394 had been previously engineered to possess FRT-F and FRT-F3 flippase recognition target sequences in the AmyB locus, where the AmyB ORF had been deleted.

Aspergillus oryzae JaL1394 was transformed with plasmid pDLHD0075 comprising the codon-optimized VaXET16 gene. Approximately 10⁷ spores from A. oryzae JaL1394 were inoculated into 100 ml of YP+2% glucose medium supplemented with 10 mM uridine in a 500 ml shake flask and incubated at 28° C. and 110 rpm overnight. Ten ml of the overnight culture were filtered in a 125 ml sterile vacuum filter, and the mycelia were washed twice with 50 ml of 0.7 M KCl-20 mM CaCl₂. The remaining liquid was removed by vacuum filtration, leaving the mat on the filter. The mycelia were resuspended in 10 ml of 0.7 M KCl-20 mM CaCl₂ and transferred to a sterile 125 ml shake flask containing 20 mg of GLUCANEX® 200 G (Novozymes Switzerland AG, Neumatt, Switzerland) per ml and 0.2 mg of chitinase (Sigma-Aldrich, St. Louis, Mo., USA) per ml in 10 ml of 0.7 M KCl-20 mM CaCl₂. The mixture was incubated at 37° C. and 100 rpm for 30-90 minutes until protoplasts were generated from the mycelia. The protoplast mixture was filtered through a sterile funnel lined with MIRACLOTH® (Calbiochem, San Diego, Calif., USA) into a sterile 50 ml plastic centrifuge tube to remove mycelial debris. The debris on the MIRACLOTH® was washed thoroughly with 10 ml of 0.7 M KCl-20 mM CaCl₂, and centrifuged at 2500 rpm for 10 minutes at 20-23° C. The supernatant was removed and the protoplast pellet was resuspended in 20 ml of 1 M sorbitol-10 mM CaCl₂−10 mM Tris-HCl (pH 6.5). This step was repeated twice, and the final protoplast pellet was resuspended in 1 M sorbitol-10 mM CaCl₂−10 mM Tris-HCl (pH 6.5) to obtain a final protoplast concentration of 2×10⁷/ml.

Protoplasts were transformed by the addition of two μg of pDLHD0075 to the bottom of a sterile 12 ml plastic centrifuge tube. One hundred μl of protoplasts were added to the tube followed by 300 μl of 60% PEG-4000 in 10 mM CaCl₂−10 mM Tris-HCl (pH 6.5). The tube was mixed gently by hand and incubated at 37° C. for 30 minutes. Five ml of 1 M sorbitol-10 mM CaCl₂−10 mM Tris-HCl (pH 6.5) were added to the transformation and the mixture was transferred onto 150 mm Minimal medium agar plates. Transformation plates were incubated at 37° C. until transformants appeared.

Single transformants were picked to new Minimal medium agar plates and cultivated at 37° C. for four days until the transformants sporulated. Fresh spores were transferred to 48-well deep-well plates containing 2 ml of YP+2% maltodextrin medium, covered with a breathable seal, and grown for 4 days at 28° C. with no shaking. After 4 days growth the culture medium for each transformant was assayed for xyloglucan endotransglycosylase activity according to Example 1 and for xyloglucan endotransglycosylase expression by SDS-PAGE.

The activity assay demonstrated that the transformants produced active xyloglucan endotransglycosylase.

SDS-PAGE was performed using a 8-16% CRITERION® Stain Free SDS-PAGE gel (Bio-Rad Laboratories, Inc., Hercules, Calif., USA), and imaging of the gel was performed with a Stain Free Imager (Bio-Rad Laboratories, Inc., Hercules, Calif., USA). SDS-PAGE revealed a band of approximately 32 kDa for the wild-type VaXET16.

### Example 10: Construction and Identification of Improved Expression Variants of Vigna angularis Xyloglucan Endotransglycosylase 16 (VaXET16)

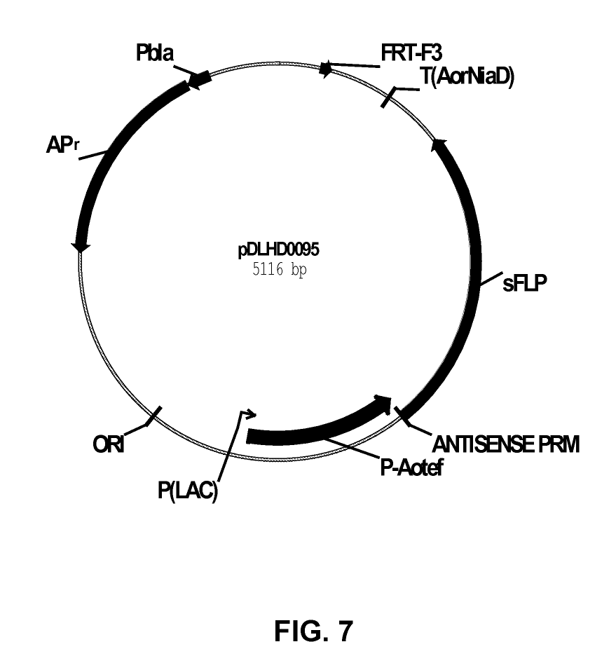

VaXET16 gene mutant libraries were constructed by site-saturation mutagenesis. The mutant libraries of the VaXET16 gene (each fragment of the library comprises a mutant VaXET16 gene plus Aspergillus nidulans orotidine 5′-phosphate decarboxylase pyrG selection marker and the FRT-F and FRT-F3 flippase recognition target sequences) were transformed into protoplasts of Aspergillus oryzae JaL1394 as described in Example 9 along with one μg of vector pDLHD0095 (FIG. 7) encoding the Saccharomyces cerevisiae 2 μm flippase ORF between the Aspergillus oryzae TEF1 promoter and Aspergillus oryzae niaD gene terminator. After 4 days of protoplast recovery at 37° C. on Minimal medium agar plates, single colonies were picked into individual wells of 48-well deep-well plates containing 2 ml of YP+2% maltodextrin medium, covered with a breathable seal, and grown for 4 days at 28° C. with no shaking. After 4 days growth the liquid culture medium was assayed for xyloglucan endotransglycosylase activity as described in Example 1, and higher activity variants were scored as expression hits.

Individual mutant strains were spore purified and cultivated again as described above to generate fresh broth for retesting relative to A. oryzae JaL1394 strain expressing the wild-type VaXET16 using the codon-optimized gene. Broths were also analyzed by SDS-PAGE as described in Example 9 for increased production of the xyloglucan endotransglycosylase protein product.

Relative improvements in expression yield over the parent gene in day 4 broths from 48-well deep-well plate cultivations for eleven characterized variants are shown in Table II below. SDS-PAGE analysis of the same broths demonstrated a VaXET band of increased intensity over wild-type VaXET for all variants, which correlated well with the relative improvements observed in the activity assay. The SDS-PAGE band for the wild-type VaXET and variants thereof was 32 kDa, except variants containing the N175S mutation had a band of approximately 37 kDa due to additional glycosylation.

TABLE II
Relative Improvement Over Parent

| VaXET16 Variant | Iodine Colorimetric Assay |
|---|---|
| Wild-Type | 1.0 |
| A40G + N175S | 1.8 |
| A40G + F183I | 2.9 |
| A40G + I53A + N175S | 3.8 |
| A40G + N175S + F183I | 1.1 |
| I10A + I53A + E102G | 1.1 |
| P30E + S51T + Y60S + T99N | 2.3 |
| A40G + E102G + Q117E | 3.1 |
| A40G + T99E + E102G + K130R | 1.2 |
| N175G + S280G | 2.7 |
| N175Q + A254E + S280E | 1.2 |
| I53V + R136W + Y157H + Y162C + | 2.9 |
| N175S | |

### Example 11: Fermentation-Scale Confirmation of Improved Expression of Vigna angularis Xyloglucan Endotransglycosylase 16 Variant (VaXET16) Genes in Aspergillus oryzae

A fermentation process was used to express the VaXET16 variants, A40G+I53A+N175S and A40G+F183I, relative to the wild-type VaXET16.

Shake flask medium was composed of 50 g of sucrose, 10 g of KH₂PO₄, 0.5 g of CaCl₂, 2 g of MgSO₄.7H₂O, 2 g of K₂SO₄, 2 g of urea, 10 g of yeast extract, 2 g of citric acid, 0.5 ml of trace metals solution, and deionized water to 1 liter. Trace metals solution was composed of 13.8 g of FeSO₄.7H₂O, 14.3 g of ZnSO₄.7H₂O, 8.5 g of MnSO₄—H₂O, 2.5 g of CuSO₄.5H₂O, 3 g of citric acid, and deionized water to 1 liter.

One hundred ml of shake flask medium were added to a 500 ml shake flask. The shake flask was inoculated with 7 ml of 0.01% TWEEN® 80 with spores scraped from a solid plate culture and incubated at 34° C. on an orbital shaker at 200 rpm for 24 hours. Fifty ml of the shake flask broth were used to inoculate a 3 liter fermentation vessel.

Fermentation batch medium was composed per liter of 10 g of yeast extract, 24 g of sucrose, 5 g of (NH₄)₂SO₄, 2 g of KH₂PO₄, 0.5 g of CaCl₂.2H₂O, 2 g of MgSO₄.7H₂O, 1 g of citric acid, 2 g of K₂SO₄, 0.5 ml of anti-foam, and 0.5 ml of trace metals solution. Trace metals solution was composed per liter of 13.8 g of FeSO₄.7H₂O, 14.3 g of ZnSO₄.7H₂O, 8.5 g of MnSO₄.H₂O, 2.5 g of CuSO₄.5H₂O, and 3 g of citric acid. Fermentation feed medium was composed of maltose.

A total of 1.8 liters of the fermentation batch medium was added to a three liter glass jacketed fermentor. Fermentation feed medium was dosed at a rate of 0 to 8.0 g/l/hr. The fermentation vessel was maintained at a temperature of 34° C. and pH was controlled to a set-point of 6.1+/−0.1. Air was added to the vessel at a rate of 1 vvm and the broth was agitated by Rushton impeller rotating at 1100 rpm. Samples were taken on days 2, 3, 4, 5, 6, and 7 of the fermentation run and centrifuged at 3000×g to remove the biomass. The supernatants were sterile filtered and stored at −20° C.

VaXET16 variant expression levels were determined relative to the wild-type codon-optimized gene by the fluorescence polarization assay (Example 3) and by SDS-PAGE analysis (Example 9).

Relative improvements in production yield over the parent gene for day 7 broths of the two variants relative to the wild-type VaXET16 are shown in Table III below. Variant A40G+I53A+N175S was produced in an amount that was 3.1× greater than the wild-type VaXET16, while variant A40G+F183I was produced in an amount that was 1.2× greater than the wild-type VaXET16. SDS-PAGE analysis of the same broths showed a VaXET band of increased intensity over wild-type VaXET for both variants which correlated well with the relative improvements observed in the activity assay. SDS-PAGE analysis of the samples taken on days 2, 3, 4, 5, 6, and 7 showed increased production of VaXET and each variant day by day with day 7 the strongest.

TABLE III
Relative Improvement Over Parent

| VaXET16 Variant | Fluorescence Polarization Assay |
|---|---|
| Wild-Type | 1.0 |
| A40G + I53A + N175S | 3.1 |
| A40G + F183I | 1.2 |

The present invention is further described by the following numbered paragraphs:

[1] A xyloglucan endotransglycosylase variant, comprising a substitution at one or more positions corresponding to positions 10, 30, 40, 51, 53, 60, 99, 102, 117, 130, 136, 157, 162, 175, 183, 254, and 280 of the full-length polypeptide of SEQ ID NO: 2, wherein the variant has xyloglucan endotransglycosylase activity and wherein the variant has at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99%, but less than 100%, sequence identity to the mature polypeptide sequence of SEQ ID NO: 2, 4, 6, 8, 10, 12, 14, 16, 18, 20, 22, 24, 26, 28, 30, 32, 34, 36, 38, 40, 42, 44, 46, 48, or 50.

[2] The variant of paragraph 1, which is a variant of a parent xyloglucan endotransglycosylase, wherein the parent is selected from the group consisting of:

(a) a polypeptide having at least 60% sequence identity to the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50;

(b) a polypeptide encoded by a polynucleotide that hybridizes under at least low stringency conditions with (i) the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, or 49, or (ii) the full-length complement of (i);

(c) a polypeptide encoded by a polynucleotide having at least 60% identity to the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, or 49; and

(d) a fragment of the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50, which has xyloglucan endotransglycosylase activity.

[3] The variant of paragraph 2, wherein the parent xyloglucan endotransglycosylase has at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, at least 99% or 100% sequence identity to the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50.

[4] The variant of paragraph 2 or 3, wherein the parent xyloglucan endotransglycosylase is encoded by a polynucleotide that hybridizes under low stringency conditions, medium stringency conditions, medium-high stringency conditions, high stringency conditions, or very high stringency conditions with (i) the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, or 49; or (ii) the full-length complement of (i).

[5] The variant of any of paragraphs 2-4, wherein the parent xyloglucan endotransglycosylase is encoded by a polynucleotide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, at least 99%, or 100% sequence identity to the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, or 49.

[6] The variant of any of paragraphs 2-5, wherein the parent xyloglucan endotransglycosylase comprises or consists of the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50.

[7] The variant of any of paragraphs 2-6, wherein the parent xyloglucan endotransglycosylase is a fragment of the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50, wherein the fragment has xyloglucan endotransglycosylase activity.

[8] The variant of any of paragraphs 2-7, which has at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99%, but less than 100%, sequence identity to the amino acid sequence of the parent xyloglucan endotransglycosylase.

[9] The variant of any of paragraphs 1-8, wherein the variant consists of at least 85%, at least 90%, or at least 95% of the amino acids of a parent.

[10] The variant of any of paragraphs 1-9, wherein the number of substitutions is 1-17, e.g., 1, 2, 3, 4, 5, 6, 7, 8, 9, 10, 11, 12, 13, 14, 15, 16, or 17 substitutions.

[11] The variant of any of paragraphs 1-10, which comprises a substitution at a position corresponding to position 10.

[12] The variant of paragraph 11, wherein the substitution is with Ala.

[13] The variant of any of paragraphs 1-12, which comprises a substitution at a position corresponding to position 30.

[14] The variant of paragraph 13, wherein the substitution is with Glu.

[15] The variant of any of paragraphs 1-14, which comprises a substitution at a position corresponding to position 40.

[16] The variant of paragraph 15, wherein the substitution is with Gly. [17] The variant of any of paragraphs 1-16, which comprises a substitution at a position corresponding to position 51.

[18] The variant of paragraph 17, wherein the substitution is with Thr.

[19] The variant of any of paragraphs 1-18, which comprises a substitution at a position corresponding to position 53.

[20] The variant of paragraph 19, wherein the substitution is with Ala or Val.

[21] The variant of any of paragraphs 1-20, which comprises a substitution at a position corresponding to position 60.

[22] The variant of paragraph 21, wherein the substitution is with Ser.

[23] The variant of any of paragraphs 1-22, which comprises a substitution at a position corresponding to position 99.

[24] The variant of paragraph 23, wherein the substitution is with Glu or Asn.

[25] The variant of any of paragraphs 1-24, which comprises a substitution at a position corresponding to position 102.

[26] The variant of paragraph 25, wherein the substitution is with Gly.

[27] The variant of any of paragraphs 1-26, which comprises a substitution at a position corresponding to position 117.

[28] The variant of paragraph 27, wherein the substitution is with Glu.

[29] The variant of any of paragraphs 1-28, which comprises a substitution at a position corresponding to position 130.

[30] The variant of paragraph 29, wherein the substitution is with Arg.

[31] The variant of any of paragraphs 1-30, which comprises a substitution at a position corresponding to position 136.

[32] The variant of paragraph 31, wherein the substitution is with Trp.

[33] The variant of any of paragraphs 1-32, which comprises a substitution at a position corresponding to position 157.

[34] The variant of paragraph 33, wherein the substitution is with His.

[35] The variant of any of paragraphs 1-34, which comprises a substitution at a position corresponding to position 162.

[36] The variant of paragraph 35, wherein the substitution is with Cys.

[37] The variant of any of paragraphs 1-36, which comprises a substitution at a position corresponding to position 175.

[38] The variant of paragraph 37, wherein the substitution is with Ser, Gly, or Gln.

[39] The variant of any of paragraphs 1-38, which comprises a substitution at a position corresponding to position 183.

[40] The variant of paragraph 39, wherein the substitution is with Ile.

[41] The variant of any of paragraphs 1-40, which comprises a substitution at a position corresponding to position 254.

[42] The variant of paragraph 41, wherein the substitution is with Glu.

[43] The variant of any of paragraphs 1-42, which comprises a substitution at a position corresponding to position 280.

[44] The variant of paragraph 43, wherein the substitution is with Gly or Glu.

[45] The variant of any of paragraphs 1-44, which comprises a substitution at two positions corresponding to any of positions 10, 30, 40, 51, 53, 60, 99, 102, 117, 130, 136, 157, 162, 175, 183, 254, and 280.

[46] The variant of any of paragraphs 1-44, which comprises a substitution at three positions corresponding to any of positions 10, 30, 40, 51, 53, 60, 99, 102, 117, 130, 136, 157, 162, 175, 183, 254, and 280.

[47] The variant of any of paragraphs 1-44, which comprises a substitution at four positions corresponding to any of positions 10, 30, 40, 51, 53, 60, 99, 102, 117, 130, 136, 157, 162, 175, 183, 254, and 280.

[48] The variant of any of paragraphs 1-44, which comprises a substitution at five positions corresponding to any of positions 10, 30, 40, 51, 53, 60, 99, 102, 117, 130, 136, 157, 162, 175, 183, 254, and 280.

[49] The variant of any of paragraphs 1-44, which comprises a substitution at six positions corresponding to any of positions 10, 30, 40, 51, 53, 60, 99, 102, 117, 130, 136, 157, 162, 175, 183, 254, and 280.

[50] The variant of any of paragraphs 1-44, which comprises a substitution at seven positions corresponding to any of positions 10, 30, 40, 51, 53, 60, 99, 102, 117, 130, 136, 157, 162, 175, 183, 254, and 280.

[51] The variant of any of paragraphs 1-44, which comprises a substitution at eight positions corresponding to any of positions 10, 30, 40, 51, 53, 60, 99, 102, 117, 130, 136, 157, 162, 175, 183, 254, and 280.

[52] The variant of any of paragraphs 1-44, which comprises a substitution at nine positions corresponding to any of positions 10, 30, 40, 51, 53, 60, 99, 102, 117, 130, 136, 157, 162, 175, 183, 254, and 280.

[53] The variant of any of paragraphs 1-44, which comprises a substitution at ten positions corresponding to any of positions 10, 30, 40, 51, 53, 60, 99, 102, 117, 130, 136, 157, 162, 175, 183, 254, and 280.

[54] The variant of any of paragraphs 1-44, which comprises a substitution at eleven positions corresponding to any of positions 10, 30, 40, 51, 53, 60, 99, 102, 117, 130, 136, 157, 162, 175, 183, 254, and 280.

[55] The variant of any of paragraphs 1-44, which comprises a substitution at twelve positions corresponding to any of positions 10, 30, 40, 51, 53, 60, 99, 102, 117, 130, 136, 157, 162, 175, 183, 254, and 280.

[56] The variant of any of paragraphs 1-44, which comprises a substitution at thirteen positions corresponding to any of positions 10, 30, 40, 51, 53, 60, 99, 102, 117, 130, 136, 157, 162, 175, 183, 254, and 280.

[57] The variant of any of paragraphs 1-44, which comprises a substitution at fourteen positions corresponding to any of positions 10, 30, 40, 51, 53, 60, 99, 102, 117, 130, 136, 157, 162, 175, 183, 254, and 280.

[58] The variant of any of paragraphs 1-44, which comprises a substitution at fifteen positions corresponding to any of positions 10, 30, 40, 51, 53, 60, 99, 102, 117, 130, 136, 157, 162, 175, 183, 254, and 280.

[59] The variant of any of paragraphs 1-44, which comprises a substitution at sixteen positions corresponding to any of positions 10, 30, 40, 51, 53, 60, 99, 102, 117, 130, 136, 157, 162, 175, 183, 254, and 280.

[60] The variant of any of paragraphs 1-44, which comprises a substitution at each position corresponding to positions 10, 30, 40, 51, 53, 60, 99, 102, 117, 130, 136, 157, 162, 175, 183, 254, and 280.

[61] The variant of any of paragraphs 1-60, which comprises one or more substitutions selected from the group consisting of I10A; P30E; A40G; S51T; I53A,V; Y60S; T99E,N; E102G; Q117E; K130R; R136W; Y157H; Y162C; N175S,G,Q; F183I; A254E; and S280G,E.

[62] The variant of paragraph 61, which comprises or consists of A40G+N175S.

[63] The variant of paragraph 61, which comprises or consists of A40G+F183I.

[64] The variant of paragraph 61, which comprises or consists of N175G+S280G.

[65] The variant of paragraph 61, which comprises or consists of A40G+I53A+N175S.

[66] The variant of paragraph 61, which comprises or consists of A40G+N175S+F183I.

[67] The variant of paragraph 61, which comprises or consists of substitutions I10A+I53A+E102G.

[68] The variant of paragraph 61, which comprises or consists of A40G+E102G+Q117E.

[69] The variant of paragraph 61, which comprises or consists of N175Q+A254E+S280E.

[70] The variant of paragraph 61, which comprises or consists of P30 E+S51T+Y60S+T99N.

[71] The variant of paragraph 61, which comprises or consists of A40G+T99E+E102G+K130R.

[72] The variant of paragraph 61, which comprises or consists of I53V+R136W+Y157H+Y162C+N175S.

[73] The variant of any of paragraphs 1-72, which has an increased expression yield relative to the parent.

[74] The variant of any of paragraphs 1-73, wherein the expression yield of the variant is increased at least 1.05, at least 1.10, at least 1.20, at least 1.30, at least 1.40, at least 1.50, at least 1.60, at least 1.70, at least 1.80, at least 1.90, at least 2, at least 2.25, at least 2.50, at least 2.75, at least 3.00, at least 3.25, at least 3.50, at least 3.75, at least 4, at least 4.25, at least 4.50, at least 4.75, at least 5, at least 6, at least 7, at least 8, at least 9, or at least 10 fold relative to the parent.

[75] An isolated polynucleotide encoding the variant of any of paragraphs 1-74.

[76] A nucleic acid construct comprising the polynucleotide of paragraph 75.

[77] An expression vector comprising the polynucleotide of paragraph 75.

[78] A recombinant host cell comprising the polynucleotide of paragraph 75.

[79] A method of producing a xyloglucan endotransglycosylase variant, comprising: cultivating the recombinant host cell of paragraph 78 under conditions suitable for expression of the variant; and

[80] The method of paragraph 79, further comprising recovering the variant.

[81] A method for obtaining a xyloglucan endotransglycosylase variant, comprising introducing into a parent xyloglucan endotransglycosylase a substitution at one or more positions corresponding to positions 10, 30, 40, 51, 53, 60, 99, 102, 117, 130, 136, 157, 162, 175, 183, 254, and 280 of the full-length polypeptide of SEQ ID NO: 2, wherein the variant has xyloglucan endotransglycosylase activity.

[82] The method of paragraph 81, further comprising recovering the variant.

[83] A method of increasing the expression yield of a xyloglucan endotransglycosylase, comprising introducing into a parent xyloglucan endotransglycosylase a substitution at one or more positions corresponding to positions 10, 30, 40, 51, 53, 60, 99, 102, 117, 130, 136, 157, 162, 175, 183, 254, and 280 of the full-length polypeptide of SEQ ID NO: 2, wherein the expression yield of the variant is increased relative to the parent.

[84] The method of paragraph 83, wherein the expression yield of the variant is increased at least 1.05, at least 1.10, at least 1.20, at least 1.30, at least 1.40, at least 1.50, at least 1.60, at least 1.70, at least 1.80, at least 1.90, at least 2, at least 2.25, at least 2.50, at least 2.75, at least 3.00, at least 3.25, at least 3.50, at least 3.75, at least 4, at least 4.25, at least 4.50, at least 4.75, at least 5, at least 6, at least 7, at least 8, at least 9, or at least 10 fold relative to the parent.

[85] The method of paragraph 84 or 85, further comprising recovering the variant.

[86] An enzyme composition comprising the variant of any of paragraphs 1-74.

[87] A whole broth formulation or cell culture composition comprising the variant of any of paragraphs 1-74.

[88] Use of the variant of any of paragraphs 1-74.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A xyloglucan endotransglycosylase variant, comprising a substitution at one or more positions corresponding to positions 10, 30, 40, 51, 53, 60, 99, 102, 117, 130, 136, 157, 162, 175, 183, 254, and 280 of the full-length polypeptide of SEQ ID NO: 2, wherein the variant has xyloglucan endotransglycosylase activity and wherein the variant has at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99%, but less than 100%, sequence identity to the mature polypeptide sequence of SEQ ID NO: 2, 4, 6, 8, 10, 12, 14, 16, 18, 20, 22, 24, 26, 28, 30, 32, 34, 36, 38, 40, 42, 44, 46, 48, or 50.

2. The variant of claim 1, which is a variant of a parent xyloglucan endotransglycosylase, wherein the parent is selected from the group consisting of:
(a) a polypeptide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, at least 99% or 100% sequence identity to the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50;
(b) a polypeptide encoded by a polynucleotide that hybridizes under low stringency conditions, medium stringency conditions, medium-high stringency conditions, high stringency conditions, or very high stringency conditions with (i) the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, or 49, or (ii) the full-length complement of (i);
(c) a polypeptide encoded by a polynucleotide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, at least 99%, or 100% sequence identity to the mature polypeptide coding sequence of SEQ ID NO: 1, 3, 5, 7, 9, 11, 13, 15, 17, 19, 21, 23, 25, 27, 29, 31, 33, 35, 37, 39, 41, 43, 45, 47, or 49; and
(d) a fragment of the mature polypeptide of SEQ ID NO: 2, 4, 6, 8, 10, 12, 14, 16, 18, 20, 22, 24, 26, 28, 30, 32, 34, 36, 38, 40, 42, 44, 46, 48, or 50, which has xyloglucan endotransglycosylase activity.

3. The variant of claim 2, which has at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, or at least 99%, but less than 100%, sequence identity to the amino acid sequence of the parent xyloglucan endotransglycosylase.

4. The variant of claim 1, wherein the number of substitutions is 1-17, e.g., 1, 2, 3, 4, 5, 6, 7, 8, 9, 10, 11, 12, 13, 14, 15, 16, or 17 substitutions.

5. The variant of claim 1, which comprises one or more substitutions selected from the group consisting of I10A; P30E; A40G; S51T; I53A,V; Y605; T99E,N; E102G; Q117E; K130R; R136W; Y157H; Y162C; N175S,G,Q; F183I; A254E; and S280G, E.

6. The variant of claim 5, which comprises or consists of A40G+N175S; A40G+F183I; N175G+S280G; A40G+I53A+N175S; A40G+N175S+F183I; I10A+I53A+E102G; A40G+E102G+Q117E; N175Q+A254E+S280E; P30E+S51T+Y605+T99N; A40G+T99E+E102G+K130R; or I53V+R136W+Y157H+Y162C+N175S.

7. The variant of claim 1, which has increased expression yield relative to the parent, wherein the expression yield of the variant is increased at least 1.05, at least 1.10, at least 1.20, at least 1.30, at least 1.40, at least 1.50, at least 1.60, at least 1.70, at least 1.80, at least 1.90, at least 2, at least 2.25, at least 2.50, at least 2.75, at least 3.00, at least 3.25, at least 3.50, at least 3.75, at least 4, at least 4.25, at least 4.50, at least 4.75, at least 5, at least 6, at least 7, at least 8, at least 9, or at least 10 fold relative to the parent.

8. An isolated polynucleotide encoding the variant of claim 1.

9. A nucleic acid construct or expression vector comprising the polynucleotide of claim 8.

10. A recombinant host cell comprising the polynucleotide of claim 8.

11. A method of producing a xyloglucan endotransglycosylase variant, comprising:
(a) cultivating the recombinant host cell of claim 10 under conditions suitable for expression of the variant; and optionally
(b) recovering the variant.

12. A method for obtaining a xyloglucan endotransglycosylase variant, comprising introducing into a parent xyloglucan endotransglycosylase a substitution at one or more positions corresponding to positions 10, 30, 40, 51, 53, 60, 99, 102, 117, 130, 136, 157, 162, 175, 183, 254, and 280 of the full-length polypeptide of SEQ ID NO: 2, wherein the variant has xyloglucan endotransglycosylase activity; and optionally recovering the variant.

13. A method of increasing the expression yield of a xyloglucan endotransglycosylase, comprising introducing into a parent xyloglucan endotransglycosylase a substitution at one or more positions corresponding to positions 10, 30, 40, 51, 53, 60, 99, 102, 117, 130, 136, 157, 162, 175, 183, 254, and 280 of the full-length polypeptide of SEQ ID NO: 2 to produce a variant, wherein the expression yield of the variant is increased relative to the parent; and optionally recovering the variant.

14. The method of claim 12, wherein the number of substitutions is 1-17, e.g., 1, 2, 3, 4, 5, 6, 7, 8, 9, 10, 11, 12, 13, 14, 15, 16, or 17 substitutions.

15. The method of claim 12, wherein the variant comprises one or more substitutions selected from the group consisting of I10A; P30E; A40G; S51T; I53A,V; Y605; T99E,N; E102G; Q117E; K130R; R136W; Y157H; Y162C; N175S,G,Q; F183I; A254E; and S280G,E.

16. The method of claim 12, wherein the variant comprises or consists of A40G+N175S; A40G+F183I; N175G+S280G; A40G+I53A+N175S; A40G+N175S+F183I; I10A+I53A+E102G; A40G+E102G+Q117E; N175Q+A254E+S280E; P30E+S51T+Y60S+T99N; A40G+T99E+E102G+K130R; or I53V+R136W+Y157H+Y162C+N175S.

17. The method of claim 13, wherein the expression yield of the variant is increased at least 1.05, at least 1.10, at least 1.20, at least 1.30, at least 1.40, at least 1.50, at least 1.60, at least 1.70, at least 1.80, at least 1.90, at least 2, at least 2.25, at least 2.50, at least 2.75, at least 3.00, at least 3.25, at least 3.50, at least 3.75, at least 4, at least 4.25, at least 4.50, at least 4.75, at least 5, at least 6, at least 7, at least 8, at least 9, or at least 10 fold relative to the parent.

18. An enzyme composition comprising the variant of claim 1.

19. A whole broth formulation or cell culture composition comprising the variant of claim 1.

20. (canceled)
